(19) United States
(12) Patent Application Publication
(10) Pub. No.: US 2015/0284694 A1
(43) Pub. Date: Oct. 8, 2015

(54) ALDEHYDE DEHYDROGENASE MUTANT, POLYNUCLEOTIDE ENCODING THE MUTANT, VECTOR AND MICROORGANISM HAVING THE POLYNUCLEOTIDE, AND METHOD OF PRODUCING 1,4-BUTANEDIOL BY USING THE SAME

(71) Applicant: Samsung Electronics Co., Ltd., Suwon-si (KR)

(72) Inventors: Yukyung Jung, Hwaseong-si (KR); Jinhwan Park, Suwon-si (KR); Jieun Kim, Suwon-si (KR); Hwayoung Cho, Hwaseongi-si (KR); Kwangmyung Cho, Seongnam-si (KR)

(21) Appl. No.: 14/596,000

(22) Filed: Jan. 13, 2015

(30) Foreign Application Priority Data
Apr. 3, 2014 (KR) 10-2014-0040064

(51) Int. Cl. C12N 9/02; C12P 7/18; C12N 9/10

(52) U.S. Cl. CPC C12N 9/0008; C12N 9/1025; C12P 7/18; C12Y 102/01003; C12Y 102/04001; C12Y 203/03001

(57) ABSTRACT

An aldehyde dehydrogenase mutant polypeptide, a recombinant microorganism including a polynucleotide encoding the polypeptide, and a method of producing 1,4-butanediol by using the same.

### RELATED APPLICATION

This application claims the benefit of Korean Patent Application No. 10-2014-0040064, filed on Apr. 3, 2014, the entire disclosure of which is hereby incorporated by reference.

### INCORPORATION BY REFERENCE OF ELECTRONICALLY SUBMITTED MATERIALS

Incorporated by reference in its entirety herein is a computer-readable nucleotide/amino acid sequence listing submitted herewith and identified as follows: One 123,597 bytes ASCII (Text) file named “718453_ST25.TXT” created Nov. 10, 2014.

### BACKGROUND

1. Field

The present disclosure relates to an aldehyde dehydrogenase mutant polypeptide, a recombinant microorganism including a polynucleotide encoding the polypeptide, and a method of producing 1,4-butanediol by using the mutant polypeptide or the microorganism.

2. Description of the Related Art

1,4-butanediol (1,4-BDO) may be used as a solvent in the manufacture of plastics, fibers, and polyurethanes. 1,4-BDO may be also converted to polytetra methylene ether glycol (PTMEG), which is a raw material for spandex fibers, via tetrahydrofuran (THF).

1,4-BDO is currently manufactured by the Reppe process using acetylene and formalin as raw materials, or by the Davy Mckee process using butane as a raw material. 1,4-BDO manufactured by chemical methods uses gas and oil-associated raw materials, and accordingly, there is a demand for alternative production methods to reduce production costs and improve environmental protection. In this regard, a method of producing 1,4-BDO by using a microorganism is suggested.

### SUMMARY

Provided is an aldehyde dehydrogenase mutant polypeptide that catalyzes conversion of 4-hydroxybutyryl-CoA to 4-hydroxybutyraldehyde and has the modified amino acid sequence of SEQ ID NO: 1 in which the amino acid at the 273^{rd} position of an amino acid sequence of SEQ ID NO: 1 is substituted with a different amino acid.

Also provided is a recombinant microorganism that includes a polynucleotide encoding the aldehyde dehydrogenase mutant polypeptide.

Further provided is a method of producing 1,4-butanediol by using the aldehyde dehydrogenase mutant polypeptide and/or the microorganism.

Additional aspects will be set forth in part in the description which follows and, in part, will be apparent from the description, or may be learned by practice of the presented embodiments.

### BRIEF DESCRIPTION OF THE DRAWINGS

These and/or other aspects will become apparent and more readily appreciated from the following description of the embodiments, taken in conjunction with the accompanying drawings in which:

FIG. 1 is a map of vector pTrc99a ald_{(M)}-cat2; and

FIG. 2 is a graph showing consumption of 4-hydroxybutyrate (4HB) and yield of 1,4-butanediol (1,4-BDO) of recombinant Escherichia coli W026 (pTrc99a ald-cat2) represented by WT, recombinant E. coli W026 (pTrc99a ald_{M1}-cat2) represented by L273I, and recombinant E. coli W026 (pTrc99a ald_{M2}-cat2) represented by L2735.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description.

An aspect of the present disclosure provides an aldehyde dehydrogenase mutant that has an activity of catalyzing the conversion of 4-hydroxybutyryl-CoA to 4-hydroxybutyraldehyde and has the modified amino acid sequence of SEQ ID NO: 1 in which the amino acid at the 273^{rd} position of SEQ ID NO: 1 is substituted with a different amino acid.

Aldehyde dehydrogenase having an amino acid sequence of SEQ ID NO: 1 may be derived from Clostridium beijerinckii. The aldehyde dehydrogenase mutant polypeptide may have a variable residue (Xaa) at the 273^{rd} position in the amino acid sequence of SEQ ID NO: 1. The aldehyde dehydrogenase mutant polypeptide may have isoleucine (Ile) or serine (Ser), instead of leucine (Leu), at the 273^{rd} position in the amino acid sequence of SEQ ID NO: 1. The mutant in which Leu is substituted with Ile at the 273^{rd} position in the amino acid sequence of SEQ ID NO: 1 may have an amino acid sequence of SEQ ID NO: 3, and the mutant in which Leu is substituted with Ser at the 273^{rd} position in the amino acid sequence of SEQ ID NO: 1 may have an amino acid sequence of SEQ ID NO: 5.

Another aspect of the present disclosure provides a polynucleotide that encodes the aldehyde dehydrogenase mutant polypeptide.

The term “polynucleotide” used herein comprehensively refers to a DNA molecule such as genomic DNA (gDNA) and complementary DNA (cDNA) and a RNA molecule. A nucleotide which is a basic building unit in a polynucleotide may include not only a natural nucleotide, but also an analogue in which a glucose or a base is modified. The polynucleotide may be an isolated polynucleotide. The polynucleotide that encodes the aldehyde dehydrogenase mutant polypeptide may be derived from C. beijerinckii.

The mutant in which Leu is substituted with Ile at the 273^{rd} position in the amino acid sequence of SEQ ID NO: 1 may be encoded by a polynucleotide having a nucleotide sequence of SEQ ID NO: 4, and the mutant in which Leu is substituted with Ser at the 273^{rd} position in the amino acid sequence of SEQ ID NO: 1 may be encoded by a polynucleotide having a nucleotide sequence of SEQ ID NO: 6.

Another aspect of the present disclosure provides a vector that includes the polynucleotide encoding the aldehyde dehydrogenase mutant polypeptide. The polynucleotide may be operably linked to a regulatory sequence, and the regulatory sequence may include a promoter, a terminator, an enhancer, or a combination thereof. The term “operably linked” used herein refers to a functional linkage between a gene to be expressed and a regulatory sequence of the gene so as to enable gene expression. The vector may further include a replication origin, a transcriptional regulatory site, a multi-cloning site, a selection marker, or a combination thereof.

Another aspect of the present disclosure provides a recombinant microorganism that includes the polynucleotide encoding the aldehyde dehydrogenase mutant.

The microorganism may include a prokaryote, a eukaryote cell, or an organism. The microorganism may include archaebacterium, eubacterium, or a eukaryotic microorganism such as yeast and fungi. The microorganism may belong to the genus Escherichia, Corynebacterium, Bacilus, Pseudomaonas, Pichia, or Saccharomyces. The microorganism may be E. coli or C. glutamicum.

In some embodiments, the microorganism that includes the polynucleotide encoding the aldehyde dehydrogenase mutant polypeptide may further have an increased activity of converting 4-hydroxybutyrate to 4-hydroxybutyryl-CoA compared to a reference microorganism.

The reference microorganism refers to a wild-type microorganism or a parental microorganism. The parental microorganism refers to a microorganism that has not undergone a subject modification (e.g., modification to introduce a polynucleotide encoding the aldehyde dehydrogenase mutant polypeptide into a microorganism) but is genetically identical to a microorganism that has not undergone a subject modification, except for the modification itself, and thus serves as a reference microorganism.

Such an increased activity may be achieved by increased expression of a polypeptide that catalyzes the conversion of 4-hydroxybutyrate to 4-hydroxybutyryl-CoA. The increased expression of the polypeptide may result from amplification of a gene that encodes the polypeptide or mutation in a regulatory site of the gene that encodes the polypeptide. The polypeptide may be 4-hydroxybutyryl CoA:acetyl-CoA transferase (Cat2). Cat2 may be an enzyme categorized as EC.2.8.3.a, and may have an amino acid sequence of SEQ ID NO: 7. The increased activity also may be achieved by introduction of a polynucleotide that encodes Cat2 into the microorganism. Such a polynucleotide may be an endogenous polynucleotide or an exogenous polynucleotide. The polynucleotide may be a polynucleotide that encodes an amino acid sequence of SEQ ID NO: 7, and may have a nucleotide sequence of SEQ ID NO: 8.

In other embodiments, the microorganism that includes the polynucleotide encoding the aldehyde dehydrogenase mutant polypeptide may have an increased activity of converting succinyl-CoA to 4-hydroxybutyrate, alpha-ketoglutarate to 4-hydroxybutyrate, or a combination thereof compared to a reference microorganism.

Such an increased activity in the conversion of succinyl-CoA to 4-hydroxybutyrate may be achieved by increased expression of a polypeptide that catalyzes conversion of succinyl-CoA to succinic semialdehyde, a polypeptide that catalyzes conversion of succinic semialdehyde to 4-hydroxybutyrate, or a combination thereof. The increased expression of the polypeptide may result from amplification of a gene that encodes the polypeptide or mutation in a regulatory site of the gene that encodes the polypeptide. The polypeptide may be an endogenous polypeptide or an exogenous polypeptide. The exogenous polypeptide may be derived from the genus Porphyromonas or the genus Clostridium.

The polypeptide that catalyzes the conversion of succinyl-CoA to succinic semialdehyde may be CoA-dependent succinate semialdehyde dehydrogenase (SucD). SucD may be an enzyme categorized as EC.1.2.1.b. SucD may have an amino acid sequence of SEQ ID NO: 9. The polypeptide that catalyzes the conversion of succinic semialdehyde to 4-hydroxybutyrate may be 4-hydroxybutyrate dehydrogenase (4Hbd). 4Hbd may be an enzyme categorized as EC.1.1.1.a. 4Hbd may have an amino acid sequence of SEQ ID NO: 11.

In addition, the increased activity in the conversion of succinyl-CoA to 4-hydroxybutyrate may be achieved by introduction of a polynucleotide that encodes SucD, a polynucleotide that encodes 4Hbd, or a combination thereof. The polynucleotide encoding SucD may be a polynucleotide that encodes an amino acid sequence of SEQ ID NO: 9. The polynucleotide encoding SucD may have a nucleotide sequence of SEQ ID NO: 10. The polynucleotide encoding 4Hbd may be a polynucleotide that encodes an amino acid sequence of SEQ ID NO: 11. The polynucleotide encoding 4Hbd may have a nucleotide sequence of SEQ ID NO: 12.

The increased activity in the conversion of alpha-ketoglutarate to 4-hydroxybutyrate may achieved by increased expression of a polypeptide that catalyzes conversion of alpha-ketoglutarate to succinic semialdehyde, a polypeptide that catalyzes conversion of succinic semialdehyde to 4-hydroxybutyrate, or a combination thereof. The polypeptide may be an endogenous polypeptide or an exogenous polypeptide. The exogenous polypeptide may be derived from the genus Porphyromonas, the genus Clostridium, or the genus Mycobacterium.

The polypeptide that catalyzes the conversion of alpha-ketoglutarate to succinic semialdehyde may be alpha-ketoglutarate decarboxylase (SucA). SucA may be an enzyme categorized as EC.4.1.1.71, and may have an amino acid sequence of SEQ ID NO: 13. The polypeptide that catalyzes the conversion of succinic semialdehyde to 4-hydroxybutyrate is defined as described above.

In addition, the increased activity in the conversion of alpha-ketoglutarate to 4-hydroxybutyrate may be achieved by introduction of a polynucleotide that encodes SucA, a polynucleotide that encodes 4Hbd, or a combination thereof. The polynucleotide encoding SucA may be a polynucleotide that encodes an amino acid sequence of SEQ ID NO: 13, and may have a nucleotide sequence of SEQ ID NO: 14. The polynucleotide encoding 4Hbd is the same as described above.

The microorganism that includes the polynucleotide encoding the aldehyde dehydrogenase mutant polypeptide may further exhibit a reduced or eliminated activity of converting pyruvate to lactate, converting pyruvate to formate, converting acetyl-CoA to ethanol, converting oxaloacetate to malate, controlling aerobic respiration, converting succinic semialdehyde to succinate, or a combination thereof. The terms “reduced,” “reduction,” “removed,” “eliminated,” and “increased” as used herein refer to a relative activity of a microorganism that is genetically engineered or modified in relation to a reference microorganism. The reference microorganism refers to a wild-type microorganism or a parental microorganism. The parental microorganism refers to a microorganism that has not undergone a subject modification (e.g., modification to reduce or eliminate the activity of converting pyruvate to lactate) but is genetically identical except for the modification, and thus serves as a reference microorganism for the modification. For example, the activity of the microorganism may be reduced by about 75% or more, about 80% or more, about 85% or more, about 90% or more, about 95% or more, or about 100% than an activity of an appropriate control.

In the microorganism, expression of a polypeptide converting pyruvate to lactate, a polypeptide converting pyruvate to formate, a polypeptide converting acetyl-CoA to ethanol, a polypeptide converting oxaloacetate to malate, a component that controls aerobic respiration, a polypeptide converting succinic semialdehyde to succinate, or a combination thereof may be reduced or eliminated. Also, in the microorganism, a polynucleotide that encodes the polypeptide converting pyruvate to lactate, a polynucleotide that encodes the polypeptide converting pyruvate to formate, a polynucleotide that encodes the polypeptide converting acetyl-CoA to ethanol, a polynucleotide that encodes the polynucleotide converting oxaloacetate to malate, a polynucleotide that encodes the component controlling aerobic respiration, a polynucleotide that encodes the polypeptide converting succinic semialdehyde to succinate, or a combination thereof may be inactivated or attenuated compared to a reference microorganism. The term “inactivated” or “inactivation” as used herein may refer to a condition of a gene that is not expressed at all, or a gene encoding an inactive protein even if expressed. The term “attenuated” or “attenuation” may refer to a condition of a gene that is expressed at a lower level compared to a reference microorganism, or a gene encoding a protein having a reduced activity compared to a reference microorganism even if expressed. The inactivation or attenuation may occur through, for example, a homologous recombination.

The polypeptide that converts pyruvate to lactate may be an enzyme categorized as EC.1.1.1.27 or EC.1.1.1.28. The polypeptide may be derived from E. coli, for example, E. coli W chromosomes. A gene that encodes the polypeptide converting pyruvate to lactate may have Gene ID: 12753486. Such a gene may be IdhA of E. coli that encodes NADH-linked lactate dehydrogenase. The IdhA gene may encode an amino acid sequence of SEQ ID NO: 15 and have a nucleotide sequence of SEQ ID NO: 16.

The polypeptide that converts pyruvate to formate may be an enzyme that reversibly converts pyruvate to formate. Such an enzyme may catalyze a reaction of pyruvate+CoA [symbol] formate+acetyl-CoA. The enzyme may be pyruvate formate lyase (Pfl) of E. coli. Pfl may be categorized as EC.2.3.1.54. A gene that encodes the polypeptide converting pyruvate to formate may have Gene ID: 2752499. Such a gene may be pflB of E. coli that encodes Pfl. The pflB gene may encode an amino acid sequence of SEQ ID NO: 17 and have a nucleotide sequence of SEQ ID NO: 18.

The polypeptide that converts acetyl-CoA to ethanol may be alcohol dehydrogenase (Adh). Adh may be an enzyme that reversibly converts acetyl-CoA to ethanol accompanied by oxidation of NADH to NAD+. Adh may be an enzyme categorized as EC.1.1.1.1. A gene that encodes the polypeptide converting acetyl-CoA to ethanol may have Gene ID: 12753141. Such a gene may be adhE of E. coli that encodes NADH-linked alcohol dehydrogenase. The adhE gene may encode an amino acid sequence of SEQ ID NO: 19, and have a nucleotide sequence of SEQ ID NO: 20.

The polypeptide that converts oxaloacetate to malate may be an enzyme that catalyzes the conversion oxaloacetate to malate accompanied by reduction of NAD+ to NADH. Such an enzyme may be malate dehydrogenase (Mdh). Mdh may be an enzyme categorized as EC 1.1.1.37. A gene that encodes the polypeptide converting oxaloacetate to malate may have GENE ID: 12697256. Such a gene may be mdh of E. coli that encodes NADH-linked malate dehydrogenase. The mdh gene may encode an amino acid sequence of SEQ ID NO: 21 and have a nucleotide sequence of SEQ ID NO: 22.

The component controlling aerobic respiration may be ArcA. ArcA may be a DNA-binding response regulator. ArcA may be a DNA-binding response regulator of two component system. The ArcA may belong to two component (ArcB-ArcA) signal-transduction system, and may form global regulation system that regulates negatively or positively expression of various operons under mutual assistance with sensory kinase ArcB of the same species. ArcA may function under micro-aerobic conditions to induce expression of a gene product which allows an activity of a core metabolic enzyme having sensitivity to low oxygen levels. Deletion in arcA/arcB genes under micro-aerobic conditions may increase specific activities of ldh, icd, gltA, mdh, and gdh genes. The arcA gene may encode an amino acid sequence of SEQ ID NO: 23 and have a nucleotide sequence of SEQ ID NO: 24.

The polypeptide that converts succinic semialdehyde to succinate may be succinate semialdehyde dehydrogenase (Ssadh). Ssadh may be an enzyme that converts succinic semialdehyde to succinate accompanied by reduction of NAD+ or NADP+ to NADH or NADPH, respectively. Ssadh may be an enzyme categorized as EC.1.2.1.24 or EC.1.2.1.16. A gene that encodes the polypeptide converting succinic semialdehyde to succinate may have Gene ID: 12695413 or 12696616. Such a gene having Gene ID: 12695413 may be sad of E. coli that encodes NAD-linked Ssadh and such a gene having Gene ID: 12696616 may be gabD of E. coli that encodes NADP-linked Ssadh. The sad gene may encode an amino acid sequence of SEQ ID NO: 25 and have a nucleotide sequence of SEQ ID NO: 26. The gabD gene may encode an amino acid sequence of SEQ ID NO: 27 and have a nucleotide sequence of SEQ ID NO: 28.

The microorganism may express a mutant subunit of foreign pyruvate dehydrogenase, a NADH insensitive citrate synthase mutant, or a combination thereof.

The subunit of foreign pyruvate dehydrogenase may be derived from Klebsiella pneumonia. The subunit may be LpdA. LpdA derived from K. pneumonia may have an amino acid sequence of SEQ ID NO: 29. The expression of the subunit of foreign pyruvate dehydrogenase may be achieved by introduction of a foreign gene. Such a gene may be lpdA derived from K. pneumonia and may have a nucleotide sequence of SEQ ID NO: 30. The mutant subunit of foreign pyruvate dehydrogenase may have an amino acid sequence in which glutamine (Glu) at the 354^{th} position in the amino acid sequence of SEQ ID NO: 29 is substituted with another, different, amino acid. The other amino acid may be lysine (Lys). The microorganism may include a polynucleotide that encodes the mutant in the subunit of foreign pyruvate dehydrogenase. The mutant in the subunit of foreign pyruvate dehydrogenase may have an amino acid sequence of SEQ ID NO: 31 and a nucleotide sequence of SEQ ID NO: 32.

The NADH insensitive citrate synthase may be GltA. GltA may have an amino acid sequence of SEQ ID NO: 33 and a nucleotide sequence of SEQ ID NO: 34. The NADH insensitive citrate synthase mutant may have an amino acid sequence in which arginine (Arg) at the 164^{th} position in the amino acid sequence of SEQ ID NO: 33 is substituted with another, different, amino acid. The other amino acid may be Leu. The microorganism may include a polynucleotide that encodes the NADH insensitive citrate synthase mutant. The citrate synthase mutant may have an amino acid sequence of SEQ ID NO: 35 and have a nucleotide sequence of SEQ ID NO: 36.

Another aspect of the present disclosure provides a method of producing 1,4-butanediol, the method including contacting the aldehyde dehydrogenase mutant polypeptide with 4-hydroxybutyryl CoA.

The contact may include culturing, and the culturing may be performed in a medium that contains 4-hydroxybutyryl CoA. The aldehyde dehydrogenase mutant polypeptide used in the method is as described herein.

Another aspect of the present disclosure provides a method of producing 1,4-butanediol, the method including culturing a microorganism that includes a polynucleotide encoding the aldehyde dehydrogenase mutant; and recovering 1,4-butanediol from the microorganism culture.

The culturing may vary according to suitable media and culturing conditions known in the art, and one of ordinary skill in the art may be able to regulate media and culturing conditions according to a selected microorganism. The culturing may include a batch culture, a continuous culture, a fed-batch culture, or a combination thereof.

The medium used herein may include various carbon sources, nitrogen sources, and trace element components. The carbon sources may be, for example, carbohydrates including glucose, sucrose, lactose, fructose, maltose, starch, and cellulose, fats including soybean oil, sunflower oil, castor oil, and coconut oil, fatty acids including palmitic acid, stearic acid, and linoleic acid, alcohol including glycerol and ethanol, organic acids including acetic acid, or a combination thereof. The culturing may be performed by using glucose as a carbon source. The nitrogen sources may be, for example, organic nitrogen sources including peptone, yeast extract, meat extract, malt extract, corn steep liquor (CSL), and soybean wheat, and inorganic nitrogen sources including urea, ammonium sulfate, ammonium chloride, ammonium phosphate, ammonium carbonate, and ammonium nitrate, or a combination thereof. The medium used herein may use phosphorus sources, such as potassium dihydrogen phosphate, dipotassium hydrogen phosphate, sodium-containing salts corresponding thereto, or metal salts including magnesium sulfate or iron sulfate. In addition, amino acids, vitamins, and appropriate precursors may be contained in the medium. The medium or individual components may be added to a culture broth in the form of a batch culture or a continuous culture.

In addition, in the middle of culturing, compounds, such as ammonium hydroxide, potassium hydroxide, ammonia, phosphoric acids, and sulfuric acids, may be added in an appropriate manner into a microbial culture broth, thereby adjusting pH of the microbial culture broth. Furthermore, in the middle of culturing, an anti-foaming agent such as fatty acid polyglycol ester may be used to inhibit generation of foams.

The culturing may be performed in aerobic or microaerobic conditions. The term “aerobic condition” used herein may refer to a culturing condition in which a medium is capable of being exchanged with oxygen-containing air. The term “micro-aerobic condition” used herein may refer to a culturing condition in which oxygen at a level less than oxygen in a normal atmosphere is dissolved in the medium. In the culturing under micro-aerobic condition, a concentration of oxygen dissolved in the medium may be about 1 to about 20%, about 1.5 to about 18%, about 2 to about 15%, about 2.5 to about 10%, or about 3 to about 8% of a saturated concentration of the oxygen. The saturated concentration may refer to a saturated concentration at a temperature at which the culturing is made. The culturing temperature may be, for example, in a range of about 20° C. to about 45° C. or about 25° C. to about 40° C.

The recovery of 1,4-butanediol may be performed according to separation and purification methods known in the art, for example, centrifugation, ion-exchange chromatography, filtration, precipitation, or a combination thereof.

Hereinafter, the present disclosure is described in greater detail with reference to embodiments. However, the embodiments are for illustrative purposes only and should in no way be interpreted to limit the scope of the present invention.

### EXAMPLE 1

### Preparation of a Microorganism having an Aldehyde Dehydrogenase (ald) Mutant Polypeptide

1.1. Preparation of a Microorganism of which a Metabolic Pathway is Manipulated for Prevention of by-product Production (e.g., Lactate, Formate, Ethanol, and Succinate) and for Cell Growth and Consumption of Carbon Source in Anaerobic Conditions

1.1.1. Deletion of IdhA, pflB, adhE, mdh, arcA, sad, and gabD Genes

According to the one-step inactivation method [refer to Warner et al., PNAS, 6; 97(12):6640-6645, 2000; lee, K. H. et al., Molecular systems biology 3, 149, 2007], deletion of IdhA, pflB, adhE, mdh, arcA, sad, and gabD genes were executed in E. coli W (ATCC 9637).

In order to execute deletion of the IdhA gene, a PCR was carried out by using a vector pMloxC [refer to Lee, K. H. et al., Molecular systems biology 3, 149 (2007)] as a template and a primer set having a nucleotide sequence of SEQ ID NO: 37 and 38. DNA fragments obtained from the PCR were subjected to electroporation in a competent cell of a W strain where λ-red recombinase was expressed, thereby preparing a mutant strain in which the IdhA gene was deleted. In order to confirm the deletion of the IdhA gene, a colony PCR was carried out by using a primer set having a nucleotide sequence of SEQ ID NO: 39 and 40. As a result, a strain E. coli W ΔldhA was obtained.

In addition, in the same manner as described above, fragments obtained from the PCR using a primer set having a nucleotide sequence of SEQ ID NO: 41 and 42 were introduced, thereby preparing a mutant strain in which the pflB gene was deleted. In order to confirm the deletion of the pflB gene, the colony PCR was carried out by using a primer set having a nucleotide sequence of SEQ ID NO: 43 and 44. As a result, a strain E. coli W ΔldhAΔpflB was obtained.

In addition, in the same manner as described above, fragments obtained from the PCR using a primer set having a nucleotide sequence of SEQ ID NO: 45 and 46 were introduced, thereby preparing a mutant strain in which the adhE gene was deleted. In order to confirm the deletion of the adhE gene, the colony PCR was carried out by using a primer set having a nucleotide sequence of SEQ ID NO: 47 and 48. As a result, a strain E. coli W ΔldhAΔpflBΔadhE was obtained.

In addition, in the same manner as described above, fragments obtained from the PCR using a primer set having a nucleotide sequence of SEQ ID NO: 49 and 50 were introduced, thereby preparing a mutant strain in which the mdh gene was deleted. In order to confirm the deletion of the mdh gene, the colony PCR was carried out by using a primer set having a nucleotide sequence of SEQ ID NO: 51 and 52. As a result, E. coli W ΔldhAΔpflBΔadhEΔmdh was obtained.

In addition, in the same manner as described above, fragments obtained from the PCR using a primer set having a nucleotide sequence of SEQ ID NO: 53 and 54 were introduced, thereby preparing a mutant strain in which the arcA gene was deleted. In order to confirm the deletion of the arcA gene, the colony PCR was carried out by using a primer set having a nucleotide sequence of SEQ ID NO: 55 and 56. As a result, E. coli W ΔldhAΔpflBΔadhEΔmdhΔarcA was obtained.

In addition, in the same manner as described above, fragments obtained from the PCR using a primer set having a nucleotide sequence of SEQ ID NO: 57 and 58 were introduced, thereby preparing a mutant strain in which the sad gene was deleted. In order to confirm the deletion of the sad gene, the colony PCR was carried out by using a primer set having a nucleotide sequence of SEQ ID NO: 59 and 60. As a result, a strain E. coli W ΔldhAΔpflBΔadhEΔmdhΔarcAΔsad was obtained.

In addition, in the same manner as described above, fragments obtained from the PCR using a primer set having a nucleotide sequence of SEQ ID NO: 61 and 62 were introduced, thereby preparing a mutant strain in which the gabD gene was deleted. In order to confirm the deletion of the gabD gene, the colony PCR was carried out by using a primer set having a nucleotide sequence of SEQ ID NO: 63 and 64. As a result, E. coli W ΔldhAΔpflBΔadhEΔmdhΔarcAΔsadΔgabD was obtained.

1.1.2. Substitution of lpdA Gene of E. coli with a Mutant of the lpdA Gene Derived from K. pneumonia

In the strain E. coli W ΔldhAΔpflBΔadhEΔmdhΔarcAΔsadΔgabD, the lpdA gene of E. coli was substituted with a mutant of the lpdA gene derived from K. pneumonia, according to the one-step inactivation method.

The mutant of the lpdA gene derived from K. pneumonia, i.e., K.lpdA (E354K), was obtained by site-direct mutagenesis using a primer set having a nucleotide sequence of SEQ ID NO: 65 and 66. A PCR was carried out by using a pSacHR06 vector [refer to US 2013-0164805] as a template and a primer set having a nucleotide sequence of SEQ ID NO: 67 and 68. DNA fragments obtained from the PCR were subjected to electroporation in a competent cell of a W strain where λ-red recombinase was expressed, thereby substituting the lpdA gene with a sacB-Km cassette. Afterwards, PCR was carried out again by using the obtained mutant K.lpdA (E354K) as a template and a primer set having a nucleotide sequence of SEQ ID NO: 69 and 70. According to the one-step inactivation method again, the site where the lpdA gene was substituted with the sacB-Km cassette was substituted with the mutant K.lpdA(E354K). In order to confirm the substituted gene, a colony PCR was carried out by using a primer set having a nucleotide sequence of SEQ ID NO: 71 and 72. As a result, a strain E. coli W ΔldhAΔpflBΔadhEΔmdhΔarcAΔsadΔgabD ΔlpdA::K.lpdA(E354 K) was obtained.

1.1.3. Introduction of a Mutant of gitA Gene of E. coli

According to the one-step inactivation method, a mutant of gltA gene of E. coli, i.e., gltA(R164L), was introduced to the strain E. coli W ΔldhAΔpflBΔadhEΔmdhΔarcAΔsadΔgabD ΔlpdA::K.lpdA(E354 K).

The mutant gltA (R164L) was obtained by inducing site-specific mutagenesis using a primer set having a nucleotide sequence of SEQ ID NO: 73 and 74. A PCR was carried out by using a vector pSacHR06 as a template and a primer set having a nucleotide sequence of SEQ ID NO: 75 and 76. DNA fragments obtained from the PCR were subjected to electroporation in a competent cell of a W strain where a λ-red recombinase was expressed, thereby substituting the gltA gene with a sacB-Km cassette. Afterwards, PCR was carried out again by using the obtained mutant gltA (R164L) as a template and a primer set having a nucleotide sequence of SEQ ID NO: 77 and 78. According to the one-step inactivation method again, the site where the gltA gene was substituted with the sacB-Km cassette was substituted with the mutant gltA (R164L). In order to confirm the substituted gene, a colony PCR was carried out by using a primer set having a nucleotide sequence of SEQ ID NO: 79 and 80. As a result, a mutant strain W ΔldhAΔpflBΔadhEΔmdhΔarcAΔsadΔgabD ΔlpdA::K.lpdA(E354K) gltA(R164L) derived from E. coli W was obtained and, and then, was named W026.

1.2. Preparation of an Expression Vector for Wild-Type Ald and Cat2

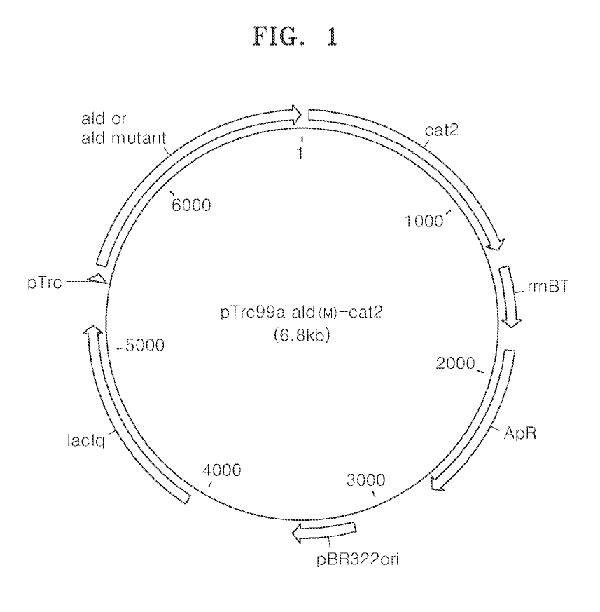

Cat2 gene derived from P. gingivali having a nucleotide sequence of SEQ ID NO: 7 and 8 and ald gene derived from C. beijerinckii having a nucleotide sequence of SEQ ID NO: 1 and 2 were prepared through gene synthesis (by COSMO Genetech Inc., Korea). The ald gene obtained therefrom was introduced by using restriction enzymes, NcoI and EcoRI, to a vector pTrc99a (AP Biotech Company), thereby preparing a vector pTrc99a ald. Then, the vector pTrc99a ald was cleaved by restriction enzymes, EcoRI and HindIII, and the cat2 gene was introduced thereto so as to prepare a vector pTrc99a ald-cat2 (see FIG. 1).

1.3. Preparation of an Expression Vector for Ald Mutant and Cat2

A PCR was carried out by using the vector pTrc99a ald-cat2 including wild-type ald having a nucleotide sequence of SEQ ID NO: 1 or 2 obtained from Example 1.2 as a template and a primer set having a nucleotide sequence of SEQ ID NO: 81 and 82, thereby preparing a vector pTrc99a ald_{M1}-cat2 that expresses an ald mutant having an amino acid sequence of SEQ ID NO: 3 in which Leu was substituted with Ile at the 273^{rd} position in the amino acid sequence (see FIG. 1). In addition, in the same manner as described above, the PCR was carried out by using a primer set having a nucleotide sequence of SEQ ID NO: 83 and 84 so as to prepare a vector pTrc99a ald_{M2}-cat2 that expresses an ald mutant having an amino acid sequence of SEQ ID NO: 5 in which Leu was substituted with Ser at the 273^{rd} position in the amino acid sequence (see FIG. 1).

1.4. Introduction of Expression Vectors to a Microorganism

Three types of the vectors, each of which included the cat2 gene and the wild-type ald or each of the two types of ald mutants prepared in Example 1.2 and Example 1.3, were each introduced to E. coli W026 of Example 1.1, according to a heat shock method (refer to Sambrook, J & Russell, D. W., New York: Cold Spring Harbor Laboratory Press, 2001), thereby preparing a strain capable of producing 1,4-BDO. Such a transgenic strain was selected and obtained from an ampicillin (100 pg/ml)-containing LB plate medium.

As a result, the recombinant strain E. coli W026 (pTrc99a ald-cat2), E. coli W026 (pTrc99a ald_{M1}-cat2), and E. coli W026 (pTrc99a ald_{M2}-cat2) were obtained. Here, the recombinant strain E. coli W026 (pTrc99a ald-cat2) to which the wild-type ald was introduced was used as a control to compare with the recombinant strains E. coli W026 (pTrc99a ald_{M1}-cat2) and E. coli W026 (pTrc99a ald_{M2}-cat2) to which two types of ald mutants were each introduced, in terms of capability of 1,4-BDO production.

### EXAMPLE 2

### Confirmation of Productivity of 1,4-BDO in the Microorganism of Example 1

The transgenic strains E. coli W026 (pTrc99a ald_{M1}-cat2) and E. coli W026 (pTrc99a ald_{M2}-cat2) of Example 1 and W026 (pTrc99a ald-cat2) as a control were inoculated in a 10 mL ampicillin (100 μg/ml)-containing LB plate medium, and the medium was pre-cultured at a temperature of 30° C. for 12 hours.

Afterwards, 0.3 mL of the pre-culture solution was inoculated to a 125 mL flask containing 30 mL of MR medium containing 15 g/L of glucose, 1 g/L of yeast extract, 10 mM of 4-hydroxybutyrate (4HB), and 100 μg/ml ampicillin, and the flask was shaken-cultured at a temperature of 30° C. at a speed of 220 rpm for 24 hours. The MR medium contained, per 1 L of distilled water, components including 6.67 g of KH₂PO₄, 4 g of (NH₄)₂HPO₄, 0.8 g of citric acid, 0.8 g of MgSO₄.7H₂O, 5 mL of trace metal solution (10 g of FeSO₄.7H₂O, 1.35 g of CaCl₂, 2.25 g of ZnSO₄.7H₂O, 0.5 g of MnSO₄.4H₂O, 1 g of CuSO₄.5H₂O, 0.106 g of (NH₄)₆Mo₇O₂₄.4H₂O, 0.23 g of Na₂B₄O₇.10H₂O, and 10 mL of 35% HCl, per 1 L of distilled water). The MR medium had a pH of 7.0 adjusted by 10N NaOH. The 4HB was synthesized through a reaction between gammabutyrolactone (Sigma-Aldrich) and NaOH. In order to induce the expression of the introduced genes, the medium was grown until optical density at 600 nanometers (OD₆₀₀) reached 0.5, and once OD₆₀₀ reached 0.5, 0.25 mM IPTG was added to the medium.

Analysis procedure for the produced 1,4-BDO is as follows: 1 mL of the 30 mL medium was centrifuged at a speed of 13,000 rpm for 30 minutes, and the supernatant obtained therefrom was centrifuged again under the same conditions. Then, 800 ul of the supernatant obtained therefrom was filtered through a 0.45 um filter to preparing a sample. 10 ul of the sample was subjected to Ultra High Performance Liquid Chromatography (UHPLC, Water) to analyze contents of 1,4-BDO, wherein UHPLC was performed by an Agilent 1100 device equipped with a Refractive index detector (RID); and a 4 mM H₂SO₄ solution was used as a mobile phase and a BIO-RAD Aminex HPX-87H Column was used as stationary phase, wherein a flow rate is 0.7 ml/min. Here, a detector and a column both had a temperature of 50° C.

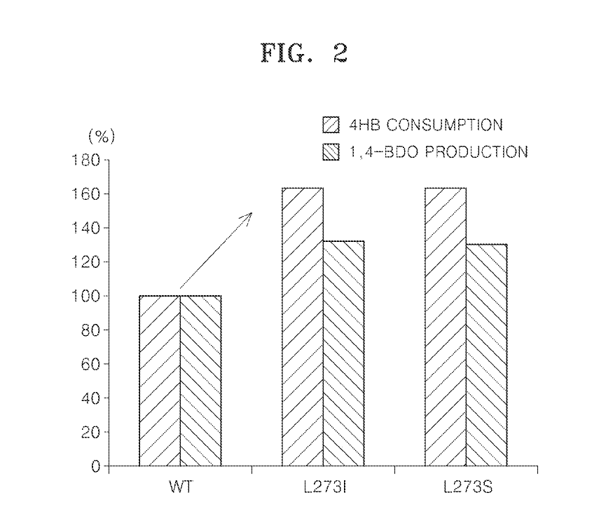

FIG. 2 depicts a graph showing consumption of 4-HB and production of 1,4-BDO of the recombinant strains E. coli W026 (pTrc99a ald-cat2) represented by WT, E. coli W026 (pTrc99a ald_{M1}-cat2) represented by L273I, and E. coli W026 (pTrc99a ald_{M2}-cat2) represented by L273S of Example 1. Here, the consumption of 4HB in E. coli W026 (pTrc99a ald_{M1}-cat2) or E. coli W026 (pTrc99a ald_{M2}-cat2) was increased about 1.64 times compared to that in E. coli W026 (pTrc99a ald-cat2) as a control, and the production of 1,4-BDO in E. coli W026 (pTrc99a ald_{M1}-cat2) or E. coli W026 (pTrc99a ald_{M2}-cat2) was increased about 1.32 times compared to that in E. coli W026 (pTrc99a ald-cat2) as a control. In this regard, it was confirmed that the catalytic activity of converting 4-hydroxybutyryl CoA to 4-hydroxybutyraldehyde of the Ald mutant having a substitution of L273I or L273S was more excellent than that of the wild-type Ald.

As described above, according to the one or more of the above embodiments of the present disclosure, 1,4-butanediol may be efficiently produced according to a method using an aldehyde dehydrogenase mutant, a polynucleotide encoding the mutant, a vector including the polynucleotide, or a microorganism including the polynucleotide.

It should be understood that the exemplary embodiments described herein should be considered in a descriptive sense only and not for purposes of limitation. Descriptions of features or aspects within each embodiment should typically be considered as available for other similar features or aspects in other embodiments.

While one or more embodiments of the present disclosure have been described with reference to the figures, it will be understood by those of ordinary skill in the art that various changes in form and details may be made therein without departing from the spirit and scope of the present disclosure as defined by the following claims.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. An aldehyde dehydrogenase mutant polypeptide comprising a mutant of SEQ ID NO: 1 in which the amino acid at the 273^{rd} position of SEQ ID NO: 1 is substituted with a different amino acid, wherein the polypeptide has a catalytic activity of converting 4-hydroxybutyryl CoA to 4-hydroxybutyraldehyde.

2. The aldehyde dehydrogenase mutant polypeptide of claim 1, wherein leucine (L) at the 273^{rd} position in SEQ ID NO: 1 is substituted with isoleucine (Ile) or serine (Ser).

3. A recombinant microorganism comprising a polynucleotide encoding the aldehyde dehydrogenase mutant polypeptide of claim 1.

4. The recombinant microorganism of claim 3, wherein the polynucleotide comprises SEQ ID NO: 4 or 6.

5. The recombinant microorganism of claim 3, wherein the polynucleotide is included in a vector.

6. The recombinant microorganism of claim 3, wherein the polynucleotide is operably linked with a regulatory sequence.

7. The recombinant microorganism of claim 3, wherein the microorganism belongs to the genus Escherichia or the genus Corynebacterium.

8. The recombinant microorganism of claim 3, wherein the microorganism has increased activity of converting 4-hydroxybutyrate into 4-hydroxybutyryl CoA compared to a reference microorganism.

9. The recombinant microorganism of claim 8, wherein the increased activity is due to increased expression of a polypeptide catalyzing the conversion of 4-hydroxybutyrate into 4-hydroxybutyryl CoA compared to a reference microorganism.

10. The recombinant microorganism of claim 8, wherein the microorganism comprises an exogenous polynucleotide encoding 4-hydroxybutyryl CoA:acetyl-CoA transferase.

11. The recombinant microorganism of claim 3, wherein the microorganism has increased an activity of converting succinyl-CoA, alpha-ketoglutarate, or a combination thereof, into 4-hydroxybutyrate compared to a reference microorganism.

12. The recombinant microorganism of claim 11, wherein the increased activity is due to increased expression of a polypeptide catalyzing the conversion of succinyl-CoA into succinic semialdehyde, a polypeptide catalyzing the conversion of alpha-ketoglutarate into succinic semialdehyde, a polypeptide catalyzing the conversion of succinic semialdehyde into 4-hydroxybutyrate, or a combination thereof, compared to a reference microorganism.

13. The recombinant microorganism of claim 11, wherein the microorganism comprises an exogenous polynucleotide encoding CoA-dependent succinic semi-aldehyde dehydrogenase, an exogenous polynucleotide encoding alpha-ketoglutarate dehydrogenase, an exogenous polynucleotide encoding 4-hydroxybutyrate semialdehyde dehydrogenase, or a combination thereof.

14. The recombinant microorganism of claim 3, wherein the microorganism has decreased activity of converting pyruvate into lactate, activity of converting pyruvate into formate, an activity of converting acetyl CoA into ethanol, activity of converting oxaloacetate into malate, activity of controlling aerobic respiration, activity of converting succinic semialdehyde to succinate, or a combination thereof, compared to a reference microorganism.

15. The recombinant microorganism of claim 14, wherein a polynucleotide encoding a polypeptide converting pyruvate into lactate, a polynucleotide encoding a polypeptide converting pyruvate into formate, a polynucleotide encoding a polypeptide converting acetyl-CoA into ethanol, a polynucleotide encoding a polypeptide converting oxaloacetate into malate, a polynucleotide encoding a component controlling aerobic respiration, a polynucleotide encoding a polypeptide converting succinic semialdehyde into succinate, or a combination thereof, is inactivated or attenuated compared to a reference microorganism.

16. The recombinant microorganism of claim 3, wherein the microorganism expresses a mutant subunit of foreign pyruvate dehydrogenase, a NADH insensitive citrate synthase mutant, or a combination thereof.

17. A method of producing 1,4-butanediol, the method comprising:
contacting the aldehyde dehydrogenase mutant polypeptide of claim 1 with 4-hydroxybutyryl CoA to provide 4-hydroxybutyraldehyde, and converting the 4-hydroxybutyraldehyde to 1,4-butanediol.

18. The method of claim 17, wherein converting 4-hydroxybutyraldehyde to 1,4-butanediol comprises contacting the 4-hydroxybutyraldehyde with alcohol dehydrogenase.

19. The method of claim 17, wherein the aldehyde dehydrogenase mutant polypeptide is produced by a recombinant microorganism comprising a polynucleotide encoding the aldehyde dehydrogenase mutant polypeptide.


SEQUENCE LISTING (84 sequences)

SEQ ID NO: 1 - protein, 468 aa, Clostridium beijerinckii
MNKDTLIPTTKDLKVKTNGENINLKNYKDNSSCFGVFENVENAISSAVHAQKILSLHYTKEQREKIITEIRKAALQNKEVLATMILEETHMGRYEDKILKHELVAKYTPGTEDLTTTAWSGDNGLTVVEMSPYGVIGAITPSTNPTETVICNSIGMIAAGNAVVFNGHPCAKKCVAFAVEMINKAIISCGGPENLVTTIKNPTMESLDAIIKHPSIKLLCGTGGPGMVKTLLNSGKKAIGAGAGNPPVIVDDTADIEKAGRSIIEGCSFDNNLPCIAEKEVFVFENVADDLISNMLKNNAVIINEDQVSKLIDLVLQKNNETQEYFINKKWVGKDAKLFLDEIDVESPSNVKCIICEVNANHPFVMTELMMPILPIVRVKDIDEAIKYAKIAEQNRKHSAYIYSKNIDNLNRFEREIDTTIFVKNAKSFAGVGYEAEGFTTFTIAGSTGEGITSARNFTRQRRCVLAG

SEQ ID NO: 2 - DNA, 1407 bp, Clostridium beijerinckii
atgaataaggatacgttgatcccgaccaccaaggatctgaaggtcaagaccaatggcgagaacatcaatcttaaaaactacaaagacaacagctcctgtttcggagtgtttgaaaatgtggaaaacgcgatctcctcagcagtacacgcgcaaaagatcctctctttgcactacactaaggaacagcgtgaaaagattatcacggagatccgcaaagcggcactgcagaacaaagaggtcctggctacaatgatcttggaggagacacatatgggtcgctacgaggacaagatcctcaagcacgagcttgttgctaagtacacccccggcaccgaggatcttaccaccaccgcctggtctggcgataatggactgaccgttgtggaaatgtccccctacggcgttatcggggcaattaccccaagcacaaacccaaccgaaaccgtgatttgtaactcgatcggaatgatcgccgcaggtaacgctgtggttttcaacggccacccatgcgcaaagaagtgcgttgcatttgccgtggagatgatcaacaaggcaattatctcatgcggtggtcctgaaaacctcgtcactaccattaagaatccaactatggagtcgcttgatgctatcattaagcacccatcgatcaagcttctctgtggaactggcggccctggcatggtcaaaacgctcctgaacagcgggaaaaaggcgattggagccggtgcaggtaatccgcccgtcatcgtggacgatacggcagatattgagaaggccggtcgttccatcatcgaaggctgctcatttgataacaacctgccgtgcattgctgagaaagaagttttcgttttcgagaacgttgccgatgaccttatttccaatatgttgaagaataatgcagtgatcatcaacgaagaccaagtttccaaactgatcgatctcgtccttcagaaaaacaacgagactcaggaatatttcattaacaagaagtgggtgggcaaagacgcaaagctgttcttggatgagattgacgtggagagcccttccaacgtcaagtgcattatctgtgaagtcaacgctaaccatcctttcgtgatgacggaattgatgatgccaatcctgccgattgttcgagtaaaagacattgacgaagctatcaagtacgcgaaaatcgccgaacagaaccgcaagcactctgcttatatctactctaagaacattgacaatctgaaccggtttgaacgggagatcgacactaccatctttgtcaaaaacgcgaaatccttcgctggcgtgggctatgaagctgagggattcaccaccttcaccattgcggggagcaccggtgaaggcatcacttctgcccgcaacttcacccgccagcgccgttgcgtactcgccggttaa

SEQ ID NO: 3 - protein, 468 aa, Artificial Sequence
  Synthetic (ald_L273I_aa)
MNKDTLIPTTKDLKVKTNGENINLKNYKDNSSCFGVFENVENAISSAVHAQKILSLHYTKEQREKIITEIRKAALQNKEVLATMILEETHMGRYEDKILKHELVAKYTPGTEDLTTTAWSGDNGLTVVEMSPYGVIGAITPSTNPTETVICNSIGMIAAGNAVVFNGHPCAKKCVAFAVEMINKAIISCGGPENLVTTIKNPTMESLDAIIKHPSIKLLCGTGGPGMVKTLLNSGKKAIGAGAGNPPVIVDDTADIEKAGRSIIEGCSFDNNIPCIAEKEVFVFENVADDLISNMLKNNAVIINEDQVSKLIDLVLQKNNETQEYFINKKWVGKDAKLFLDEIDVESPSNVKCIICEVNANHPFVMTELMMPILPIVRVKDIDEAIKYAKIAEQNRKHSAYIYSKNIDNLNRFEREIDTTIFVKNAKSFAGVGYEAEGFTTFTIAGSTGEGITSARNFTRQRRCVLAG

SEQ ID NO: 4 - DNA, 1407 bp, Artificial Sequence
  Synthetic (ald_L273I_nt)
atgaataaggatacgttgatcccgaccaccaaggatctgaaggtcaagaccaatggcgagaacatcaatcttaaaaactacaaagacaacagctcctgtttcggagtgtttgaaaatgtggaaaacgcgatctcctcagcagtacacgcgcaaaagatcctctctttgcactacactaaggaacagcgtgaaaagattatcacggagatccgcaaagcggcactgcagaacaaagaggtcctggctacaatgatcttggaggagacacatatgggtcgctacgaggacaagatcctcaagcacgagcttgttgctaagtacacccccggcaccgaggatcttaccaccaccgcctggtctggcgataatggactgaccgttgtggaaatgtccccctacggcgttatcggggcaattaccccaagcacaaacccaaccgaaaccgtgatttgtaactcgatcggaatgatcgccgcaggtaacgctgtggttttcaacggccacccatgcgcaaagaagtgcgttgcatttgccgtggagatgatcaacaaggcaattatctcatgcggtggtcctgaaaacctcgtcactaccattaagaatccaactatggagtcgcttgatgctatcattaagcacccatcgatcaagcttctctgtggaactggcggccctggcatggtcaaaacgctcctgaacagcgggaaaaaggcgattggagccggtgcaggtaatccgcccgtcatcgtggacgatacggcagatattgagaaggccggtcgttccatcatcgaaggctgctcatttgataacaacatcccgtgcattgctgagaaagaagttttcgttttcgagaacgttgccgatgaccttatttccaatatgttgaagaataatgcagtgatcatcaacgaagaccaagtttccaaactgatcgatctcgtccttcagaaaaacaacgagactcaggaatatttcattaacaagaagtgggtgggcaaagacgcaaagctgttcttggatgagattgacgtggagagcccttccaacgtcaagtgcattatctgtgaagtcaacgctaaccatcctttcgtgatgacggaattgatgatgccaatcctgccgattgttcgagtaaaagacattgacgaagctatcaagtacgcgaaaatcgccgaacagaaccgcaagcactctgcttatatctactctaagaacattgacaatctgaaccggtttgaacgggagatcgacactaccatctttgtcaaaaacgcgaaatccttcgctggcgtgggctatgaagctgagggattcaccaccttcaccattgcggggagcaccggtgaaggcatcacttctgcccgcaacttcacccgccagcgccgttgcgtactcgccggttaa

SEQ ID NO: 5 - protein, 468 aa, Artificial Sequence
  Synthetic (ald_L273S_aa)
MNKDTLIPTTKDLKVKTNGENINLKNYKDNSSCFGVFENVENAISSAVHAQKILSLHYTKEQREKIITEIRKAALQNKEVLATMILEETHMGRYEDKILKHELVAKYTPGTEDLTTTAWSGDNGLTVVEMSPYGVIGAITPSTNPTETVICNSIGMIAAGNAVVFNGHPCAKKCVAFAVEMINKAIISCGGPENLVTTIKNPTMESLDAIIKHPSIKLLCGTGGPGMVKTLLNSGKKAIGAGAGNPPVIVDDTADIEKAGRSIIEGCSFDNNSPCIAEKEVFVFENVADDLISNMLKNNAVIINEDQVSKLIDLVLQKNNETQEYFINKKWVGKDAKLFLDEIDVESPSNVKCIICEVNANHPFVMTELMMPILPIVRVKDIDEAIKYAKIAEQNRKHSAYIYSKNIDNLNRFEREIDTTIFVKNAKSFAGVGYEAEGFTTFTIAGSTGEGITSARNFTRQRRCVLAG

SEQ ID NO: 6 - DNA, 1407 bp, Artificial Sequence
  Synthetic (ald_L273S_nt)
atgaataaggatacgttgatcccgaccaccaaggatctgaaggtcaagaccaatggcgagaacatcaatcttaaaaactacaaagacaacagctcctgtttcggagtgtttgaaaatgtggaaaacgcgatctcctcagcagtacacgcgcaaaagatcctctctttgcactacactaaggaacagcgtgaaaagattatcacggagatccgcaaagcggcactgcagaacaaagaggtcctggctacaatgatcttggaggagacacatatgggtcgctacgaggacaagatcctcaagcacgagcttgttgctaagtacacccccggcaccgaggatcttaccaccaccgcctggtctggcgataatggactgaccgttgtggaaatgtccccctacggcgttatcggggcaattaccccaagcacaaacccaaccgaaaccgtgatttgtaactcgatcggaatgatcgccgcaggtaacgctgtggttttcaacggccacccatgcgcaaagaagtgcgttgcatttgccgtggagatgatcaacaaggcaattatctcatgcggtggtcctgaaaacctcgtcactaccattaagaatccaactatggagtcgcttgatgctatcattaagcacccatcgatcaagcttctctgtggaactggcggccctggcatggtcaaaacgctcctgaacagcgggaaaaaggcgattggagccggtgcaggtaatccgcccgtcatcgtggacgatacggcagatattgagaaggccggtcgttccatcatcgaaggctgctcatttgataacaactccccgtgcattgctgagaaagaagttttcgttttcgagaacgttgccgatgaccttatttccaatatgttgaagaataatgcagtgatcatcaacgaagaccaagtttccaaactgatcgatctcgtccttcagaaaaacaacgagactcaggaatatttcattaacaagaagtgggtgggcaaagacgcaaagctgttcttggatgagattgacgtggagagcccttccaacgtcaagtgcattatctgtgaagtcaacgctaaccatcctttcgtgatgacggaattgatgatgccaatcctgccgattgttcgagtaaaagacattgacgaagctatcaagtacgcgaaaatcgccgaacagaaccgcaagcactctgcttatatctactctaagaacattgacaatctgaaccggtttgaacgggagatcgacactaccatctttgtcaaaaacgcgaaatccttcgctggcgtgggctatgaagctgagggattcaccaccttcaccattgcggggagcaccggtgaaggcatcacttctgcccgcaacttcacccgccagcgccgttgcgtactcgccggttaa

SEQ ID NO: 7 - protein, 431 aa, Porphyromonas gingivalis
MKDVLAEYASRIVSAEEAVKHIKNGERVALSHAAGVPQSCVDALVQQADLFQNVEIYHMLCLGEGKYMAPEMAPHFRHITNFVGGNSRKAVEENRADFIPVFFYEVPSMIRKDILHIDVAIVQLSMPDENGYCSFGVSCDYSKPAAESAHLVIGEINRQMPYVHGDNLIHISKLDYIVMADYPIYSLAKPKIGEVEEAIGRNCAELIEDGATLQLGIGAIPDAALLFLKDKKDLGIHTEMFSDGVVELVRSGVITGKKKTLHPGKMVATFLMGSEDVYHFIDKNPDVELYPVDYVNDPRVIAQNDNMVSINSCIEIDLMGQVVSECIGSKQFSGTGGQVDYVRGAAWSKNGKSIMAIPSTAKNGTASRIVPIIAEGAAVTTLRNEVDYVVTEYGIAQLKGKSLRQRAEALIAIAHPDFREELTKHLRKRFG

SEQ ID NO: 8 - DNA, 1296 bp, Porphyromonas gingivalis
atgaaagacgtgttagcggaatatgcctcccgaattgtttcggccgaagaggcagtcaaacatatcaaaaatggagagcgtgtcgctttatcacatgctgccggagttcctcagagttgtgttgacgcactggtgcaacaggcggacctgtttcagaatgtggagatttaccacatgctgtgtctcggcgaaggaaaatatatggcacctgaaatggcccctcacttccggcacataaccaattttgttggtggtaactctcgtaaagcagtggaggaaaatagagccgacttcattccggtattcttttatgaagtgccatcaatgattcggaaagatatccttcatatagatgtggccattgtccaactctcaatgccagatgagaatggttactgcagctttggcgtatcttgcgattatagcaaaccggcggcggaatcggcgcatttagttattggggaaatcaaccgtcagatgccatatgtgcatggtgacaacttgattcacatatcgaagttggattacatcgtgatggcggattacccaatttattctctggcgaagcccaaaatcggagaagtagaggaagctatcggccgtaactgtgccgagcttattgaagatggtgccaccctacagctgggtatcggcgcgattccggatgcagctctgctgtttctgaaggacaaaaaagatctggggattcatactgaaatgttctccgatggcgttgttgaactggtgcgcagtggtgtaattactggaaaaaaaaagacattgcatcccggtaagatggtcgcgacgtttcttatgggatcagaagacgtgtatcatttcatcgacaagaatccggatgtggaactgtatccggttgattacgtcaatgatccgagggttatcgctcagaatgataatatggtcagcatcaatagctgtatcgagatcgatctaatgggccaagtggtgagcgagtgcataggctccaaacagtttagtggcaccgggggtcaagtagattatgtccgcggggcagcttggtctaaaaacggcaaaagcatcatggcaattccctcaacagccaaaaacggtactgcatctcggatagttcctataattgcagagggcgctgctgtaacaaccctccgcaacgaagtcgactacgttgttacggaatatgggatagcacagttaaaaggtaagagtttgcgtcagcgcgcagaagctcttattgcgatagcccacccggactttagagaggaactgacgaagcatctgcgcaaacgttttggttaa

SEQ ID NO: 9 - protein, 451 aa, Porphyromonas gingivalis
MEIKEMVSLARKAQKEYQATHNQEAVDNICRAAAKVIYENAAILAREAVDETGMGVYEHKVAKNQGKSKGVWYNLHNKKSIGILNIDERTGMIEIAKPIGVVGAVTPTTNPIVTPMSNIIFALKTCNAIIIAPHPRSKKCSAHAVRLIKEAIAPFNVPEGMVQIIEEPSIEKTQELMGAVDVVVATGGMGMVKSAYSSGKPSFGVGAGNVQVIVDSNIDFEAAAEKIITGRAFDNGIICSGEQSIIYNEADKEAVFTAFRNHGAYFCDEAEGDRARAAIFENGAIAKDVVGQSVAFIAKKANINIPEGTRILVVEARGVGAEDVICKEKMCPVMCALSYKHFEEGVEIARTNLANEGNGHTCAIHSNNQAHIILAGSELTVSRIVVNAPSATTAGGHIQNGLAVTNTLGCGSWGNNSISENFTYKHLLNISRIAPLNSSIHIPDDKEIWEL

SEQ ID NO: 10 - DNA, 1356 bp, Porphyromonas gingivalis
atggaaataaaagagatggtgtcgttggcaaggaaagctcagaaggaatatcaagcgacccataatcaagaagcagttgataacatttgccgagctgcagcaaaagtgatttatgaaaatgcagctatactggctcgcgaagcagtagacgaaaccggcatgggcgtatatgaacataaagtggccaagaatcaggggaaatccaaaggcgtctggtacaatttgcacaataaaaaatcgatcggtatcttaaatatagacgagagaaccgggatgatcgagatagcaaaacctatcggggttgttggagccgtaaccccgacgacaaacccgattgtgactccaatgagcaacatcatttttgcccttaagacatgcaatgccattattatcgccccacatcccagatccaaaaaatgctcagcacatgcagttcgtctgataaaggaagcaatcgctccgtttaatgtcccggagggaatggttcagatcattgaagagcccagcatcgagaaaactcaggaactaatgggcgccgtggatgtggtagttgcgacgggtggtatgggtatggtgaaatctgcatattcttcagggaagccttcttttggtgtaggagccggtaacgttcaagtgatcgtggatagtaatatcgattttgaagctgcggcagaaaaaattatcaccggccgtgctttcgacaatgggatcatctgttcaggcgaacagagtatcatctacaacgaagctgacaaggaagctgtcttcacagccttccgcaaccatggtgcatatttttgtgatgaagcggagggagatcgggcccgtgctgcgatttttgagaatggcgccatcgcgaaagatgtagtcggccagagcgttgcctttatcgcgaagaaagcaaatatcaatataccggagggtacccgtattctggttgttgaagctcgcggcgtcggagcagaggatgtcatatgtaaggaaaaaatgtgtccagttatgtgcgccttaagctacaagcacttcgaggaaggtgtagaaatcgcacgtacgaacttggccaacgaaggtaacggccatacctgtgcgatccattccaacaatcaggcgcatatcatactggcaggttcagaactgacggtttcgcggatcgtggtcaatgcgccgagtgccactacagcaggcggtcacatccaaaatggtctggcagtgacaaatacgctcggatgcgggagttggggtaataactctatctccgagaactttacttataaacacctgttaaacattagccgcatagcgccgcttaattcaagcattcacattcctgatgacaaagagatctgggaactctaa

SEQ ID NO: 11 - protein, 371 aa, Porphyromonas gingivalis
MQLFKLKSVTHHFDTFAEFAKEFCLGERDLVITNEFIYEPYMKACQLPCHFVMQEKYGQGEPSDEMMNNILADIRNIQFDRVIGIGGGTVIDISKLFVLKGLNDVLDAFDRKIPLIKEKELIIVPTTCGTGSEVTNISIAEIKSRHTKMGLADDAIVADHAIIIPELLKSLPFHFYACSAIDALIHAIESYVSPKASPYSRLFSEAAWDIILEVFKKIAEHGPEYRFEKLGEMIMASNYAGIAFGNAGVGAVHALSYPLGGNYHVPHGEANYQFFTEVFKVYQKKNPFGYIVELNWKLSKILNCQPEYVYPKLDELLGCLLTKKPLHEYGMKDEEVRGFAESVLKTQQRLLANNYVELTVDEIEGIYRRLY

SEQ ID NO: 12 - DNA, 1116 bp, Porphyromonas gingivalis
atgcaactgttcaaactgaaatcagtcacacatcacttcgatactttcgcggaatttgccaaagagttctgtcttggagaacgtgatttagtaattaccaacgaattcatttacgaaccgtatatgaaggcatgtcagttgccctgccattttgttatgcaggagaaatatgggcaaggcgagccatctgacgagatgatgaataacatcttggcagacatccgtaatatccagtttgaccgcgtgatcggtattgggggtggtacggttattgacatctcgaaattatttgtgctgaaaggactaaatgatgtgctcgatgcgttcgatcgcaagataccgctgattaaagagaaagaactgatcattgtgcccaccacatgcgggacgggtagcgaggtgacgaatatttcgatcgcggagatcaaaagccgtcataccaaaatgggtttggctgacgatgctattgttgcagaccacgcgatcatcataccagagcttctgaaaagcctgccgttccatttttatgcatgcagtgcaatagatgctctgatccatgccatcgagtcatatgtttctcctaaagccagtccatattctcgtctgttcagtgaggcggcatgggatattatcctggaggtattcaagaaaatagccgaacacggccctgaataccgctttgagaagctgggagaaatgatcatggcctccaactatgctggtatagccttcgggaatgcaggcgtgggtgccgttcacgctctaagctatccattgggaggcaattatcatgtgccgcatggcgaggctaactatcagttttttacagaggtctttaaagtataccaaaagaaaaatcctttcggctatatagtcgaactcaactggaagctgtccaagattctgaactgtcagcctgaatacgtctatccgaaactggatgagttactcggctgtcttctgaccaaaaaaccgctgcacgaatacggcatgaaagatgaagaggtacgtggatttgcggaatcagtgcttaagactcagcagcggttgctcgcgaataattatgttgagcttactgttgatgaaattgaaggtatctacagacgactgtactaa

SEQ ID NO: 13 - protein, 1214 aa, Mycobacterium bovis
MYRKFRDDPSSVDPSWHEFLVDYSPEPTSQPAAEPTRVTSPLVAERAAAAAPQAPPKPADTAAAGNGVVAALAAKTAVPPPAEGDEVAVLRGAAAAVVKNMSASLEVPTATSVRAVPAKLLIDNRIVINNQLKRTRGGKISFTHLLGYALVQAVKKFPNMNRHYTEVDGKPTAVTPAHTNLGLAIDLQGKDGKRSLVVAGIKRCETMRFAQFVTAYEDIVRRARDGKLTTEDFAGVTISLTNPGTIGTVHSVPRLMPGQGAIIGVGAMEYPAEFQGASEERIAELGIGKLITLTSTYDHRIIQGAESGDFLRTIHELLLSDGFWDEVFRELSIPYLPVRWSTDNPDSIVDKNARVMNLIAAYRNRGHLMADTDPLRLDKARFRSHPDLEVLTHGLTLWDLDRVFKVDGFAGAQYKKLRDVLGLLRDAYCRHIGVEYAHILDPEQKEWLEQRVETKHVKPTVAQQKYILSKLNAAEAFETFLQTKYVGQKRFSLEGAESVIPMMDAAIDQCAEHGLDEVVIGMPHRGRLNVLANIVGKPYSQIFTEFEGNLNPSQAHGSGDVKYHLGATGLYLQMFGDNDIQVSLTANPSHLEAVDPVLEGLVRAKQDLLDHGSIDSDGQRAFSVVPLMLHGDAAFAGQGVVAETLNLANLPGYRVGGTIHIIVNNQIGFTTAPEYSRSSEYCTDVAKMIGAPIFHVNGDDPEACVWVARLAVDFRQRFKKDVVIDMLCYRRRGHNEGDDPSMTNPYMYDVVDTKRGARKSYTEALIGRGDISMKEAEDALRDYQGQLERVFNEVRELEKHGVQPSESVESDQMIPAGLATAVDKSLLARIGDAFLALPNGFTAHPRVQPVLEKRREMAYEGKIDWAFGELLALGSLVAEGKLVRLSGQDSRRGTFSQRHSVLIDRHTGEEFTPLQLLATNSDGSPTGGKFLVYDSPLSEYAAVGFEYGYTVGNPDAVVLWEAQFGDFVNGAQSIIDEFISSGEAKWGQLSNVVLLLPHGHEGQGPDHTSARIERFLQLWAEGSMTIAMPSTPSNYFHLLRRHALDGIQRPLIVFTPKSMLRHKAAVSEIKDFTEIKFRSVLEEPTYEDGIGDRNKVSRILLTSGKLYYELAARKAKDNRNDLAIVRLEQLAPLPRRRLRETLDRYENVKEFFWVQEEPANQGAWPRFGLELPELLPDKLAGIKRISRRAMSAPSSGSSKVHAVEQQEILDEAFG

SEQ ID NO: 14 - DNA, 3645 bp, Mycobacterium bovis
atgtaccgtaaattccgtgatgacccgtcttctgttgatccgtcttggcacgaatttctggtcgattactccccggaaccaacttcccagccggccgctgaaccgacccgcgttacgtcccctctggtcgcggaacgtgcagctgcggcagcaccgcaggcgccaccaaaacctgctgataccgctgcagctggtaatggtgtggttgctgcactggctgctaaaacggctgttccgccgcctgctgaaggtgatgaagtggccgtgctgcgtggtgcggcagccgcggtcgtcaaaaacatgagcgcgtctctggaagtgccgacggcgaccagcgtgcgcgcggttccagcgaaactgctgattgataatcgtattgtgatcaacaaccagctgaaacgtacccgtggtggcaaaattagctttacccacctgctgggttatgccctggtgcaggcggtgaagaaattcccgaacatgaaccgtcactacaccgaagtcgacggtaaaccgactgccgtgaccccggcacacaccaacctgggcctggcaattgacctgcagggcaaggatggcaagcgttccctggtagtagctggtattaaacgttgcgaaaccatgcgctttgcacagttcgtaaccgcgtacgaagatatcgtacgtcgcgcacgtgatggcaaactgactaccgaagacttcgcgggtgtgaccatttccctgaccaacccgggcaccatcggtactgtacatagcgtaccacgtctgatgccgggtcagggtgcgattatcggcgttggtgctatggagtatccggccgagtttcagggtgcttccgaagagcgtatcgcggaactgggtattggtaaactgattaccctgacgagcacctacgaccaccgcatcatccagggcgccgaaagcggtgacttcctgcgtaccatccatgaactgctgctgtccgatggtttctgggatgaagtcttccgcgaactgtctattccgtacctgccggtccgttggtccaccgataacccggattctattgtagacaaaaacgcccgcgttatgaacctgatcgcagcgtatcgtaatcgtggccacctgatggcagacacggaccctctgcgtctggacaaagcgcgttttcgcagccacccggacctggaagttctgactcatggcctgactctgtgggatctggatcgcgtatttaaagtggatggctttgcaggtgcccagtacaagaaactgcgtgatgttctgggcctgctgcgtgacgcctattgccgccatattggtgttgaatacgcgcacatcctggacccagagcagaaagaatggctggagcagcgtgtggaaaccaaacacgttaagccgaccgtagcgcagcagaaatacatcctgtctaagctgaacgctgccgaggctttcgaaacctttctgcagacgaaatatgttggtcagaaacgcttctccctggagggtgcagaatctgtgatcccgatgatggatgctgcgatcgaccagtgcgctgaacacggcctggacgaggtagtgatcggtatgccgcaccgtggccgtctgaacgttctggctaacatcgttggtaaaccgtacagccagatctttactgaattcgaaggcaacctgaacccgtcccaggctcatggttccggcgacgtgaaataccatctgggcgcaactggtctgtacctgcagatgttcggtgataatgacatccaggtatctctgaccgctaatccgtcccacctggaagcggttgacccggtactggaaggcctggttcgtgcaaaacaagatctgctggaccacggtagcatcgattctgacggtcagcgtgccttctctgtggttccgctgatgctgcacggcgatgcggcttttgcaggccagggtgttgttgctgaaacgctgaacctggcgaacctgccgggctaccgtgttggtggcactatccatatcatcgttaacaaccagatcggcttcacgaccgcgccggaatactctcgctctagcgaatactgcactgatgtggctaagatgattggcgccccaatcttccacgttaacggtgacgacccggaagcgtgtgtgtgggttgcccgtctggctgtggatttccgtcaacgtttcaaaaaggacgttgttatcgacatgctgtgttaccgtcgtcgcggccacaacgaaggcgacgatccgagcatgactaacccttacatgtacgatgtagttgacaccaaacgtggcgcacgtaaaagctatactgaagcgctgatcggtcgtggtgatatctctatgaaagaagcagaagacgcactgcgcgactatcaaggccaactggaacgcgttttcaacgaagttcgcgagctggagaaacacggtgtccaacctagcgaatctgtggaatctgaccagatgatcccggcgggtctggcaactgcagtggacaaaagcctgctggcacgtattggcgacgcgttcctggctctgccgaacggtttcactgcacacccacgtgtacagccggttctggaaaaacgtcgtgaaatggcctacgaaggtaaaatcgactgggcttttggtgagctgctggcgctgggctccctggttgcggagggtaaactggtccgtctgagcggtcaagattctcgtcgtggtactttcagccagcgtcactctgtgctgatcgatcgtcacacgggtgaagaattcaccccgctgcaactgctggcgaccaactccgatggctctcctaccggtggtaaattcctggtatacgactctccactgtctgaatatgctgcagttggcttcgaatacggttacactgttggtaacccggacgctgttgtgctgtgggaagctcagttcggcgacttcgtaaatggcgcgcagtccatcattgacgaattcatttcctctggcgaagcgaaatggggccagctgtccaacgtcgtgctgctgctgccacacggccatgaaggtcagggtccggatcatacttctgcgcgcatcgagcgtttcctgcagctgtgggccgagggctccatgaccatcgccatgccgtccaccccgtctaattattttcacctgctgcgccgtcacgcgctggacggtatccagcgcccgctgattgttttcaccccgaaatccatgctgcgccacaaagcggcagtcagcgagattaaagatttcaccgaaatcaaattccgctccgtcctggaagaaccgacctatgaagacggcatcggtgaccgcaacaaggtaagccgcattctgctgacctccggcaaactgtattacgagctggcagctcgcaaggcgaaggataaccgcaacgatctggcaatcgtgcgcctggaacagctggcgccgctgccgcgtcgccgtctgcgtgaaaccctggatcgctatgaaaacgtaaaagagttcttctgggttcaagaagagccggcaaaccagggcgcttggccgcgttttggcctggagctgccggagctgctgccggacaagctggccggtatcaaacgtatctcccgtcgtgctatgagcgccccttctagcggttcttctaaagttcatgctgttgaacagcaagaaatcctggacgaagcgttcggctaa

SEQ ID NO: 15 - protein, 329 aa, Escherichia coli
MKLAVYSTKQYDKKYLQQVNESFGFELEFFDFLLTEKTAKTANGCEAVCIFVNDDGSRPVLEELKKHGVKYIALRCAGFNNVDLDAAKELGLKVVRVPAYDPEAVAEHAIGMMMTLNRRIHRAYQRTRDANFSLEGLTGFTMYGKTAGVIGTGKIGVAMLRILKGFGMRLLAFDPYPSAAALELGVEYVDLPTLFSESDVISLHCPLTPENYHLLNEAAFDQMKNGVMIVNTSRGALIDSQAAIEALKNQKIGSLGMDVYENERDLFFEDKSNDVIQDDVFRRLSACHNVLFTGHQAFLTAEALTSISQTTLQNLSNLEKGETCPNELV

SEQ ID NO: 16 - DNA, 990 bp, Escherichia coli
atgaaactcgccgtttatagcacaaaacagtacgacaagaagtacctgcaacaggtgaacgagtcctttggctttgagctggaattttttgactttctgctgacggaaaaaaccgctaaaactgccaatggctgcgaagcggtatgtattttcgtaaacgatgacggcagccgcccggtgctggaagagctgaaaaagcacggcgttaaatatatcgccctgcgctgtgccggtttcaataacgtcgaccttgacgcggcaaaagaactggggctgaaagtagtccgtgttccagcctatgatccagaggccgttgctgaacacgccatcggtatgatgatgacgctgaaccgccgtattcaccgcgcgtatcagcgtacccgtgacgctaacttctctctggaaggtctgaccggctttactatgtatggcaaaacggcaggcgttatcggtaccggtaaaatcggtgtggcgatgctgcgcattctgaaaggttttggtatgcgtctgctggcgttcgatccgtatccaagtgcagcggcgctggaactcggtgtggagtatgtcgatctgccaaccctgttctctgaatcagacgttatctctctgcactgcccgctgacaccggaaaactaccatctgttgaacgaagccgccttcgatcagatgaaaaatggcgtgatgatcgtcaataccagtcgcggtgcattgattgattctcaggcagcaattgaagcgctgaaaaatcagaaaattggttcgttgggtatggacgtgtatgagaacgaacgcgatctattctttgaagataaatccaacgacgtaattcaggatgacgtattccgtcgcctgtctgcctgccacaacgtgctatttaccgggcaccaggcattcctgacagcagaagctctgaccagtatttctcagactacgctgcaaaacttaagcaatctggaaaaaggcgaaacctgcccgaacgaactggtttaa

SEQ ID NO: 17 - protein, 760 aa, Escherichia coli
MSELNEKLATAWEGFTKGDWQNEVNVRDFIQKNYTPYEGDESFLAGATEATTTLWDKVMEGVKLENRTHAPVDFDTAVASTITSHDAGYINKALEKVVGLQTEAPLKRALIPFGGIKMIEGSCKAYNRELDPMIKKIFTEYRKTHNQGVFDVYTPDILRCRKSGVLTGLPDAYGRGRIIGDYRRVALYGIDYLMKDKYAQFTSLQADLENGVNLEQTIRLREEIAEQHRALGQMKEMAAKYGYDISGPATNAQEAIQWTYFGYLAAVKSQNGAAMSFGRTSTFLDVYIERDLKAGKITEQEAQEMVDHLVMKLRMVRFLRTPEYDELFSGDPIWATESIGGMGLDGRTLVTKNSFRFLNTLYTMGPSPEPNMTILWSEKLPLNFKKFAAKVSIDTSSLQYENDDLMRPDFNNDDYAIACCVSPMIVGKQMQFFGARANLAKTMLYAINGGVDEKLKMQVGPKSEPIKGDVLNYDEVMERMDHFMDWLAKQYITALNIIHYMHDKYSYEASLMALHDRDVIRTMACGIAGLSVAADSLSAIKYAKVKPIRDEDGLAIDFEIEGEYPQFGNNDPRVDDLAVDLVERFMKKIQKLHTYRDAIPTQSVLTITSNVVYGKKTGNTPDGRRAGAPFGPGANPMHGRDQKGAVASLTSVAKLPFAYAKDGISYTFSIVPNALGKDDEVRKTNLAGLMDGYFHHEASIEGGQHLNVNVMNREMLLDAMENPEKYPQLTIRVSGYAVRFNSLTKEQQQDVITRTFTQSM

SEQ ID NO: 18 - DNA, 2283 bp, Escherichia coli
atgtccgagcttaatgaaaagttagccacagcctgggaaggttttaccaaaggtgactggcagaatgaagtaaacgtccgtgacttcattcagaaaaactacactccgtacgagggtgacgagtccttcctggctggcgctactgaagcgaccaccaccctgtgggacaaagtaatggaaggcgttaaactggaaaaccgcactcacgcgccagttgactttgacaccgctgttgcttccaccatcacctctcacgacgctggctacatcaacaaagcgttggaaaaagttgttggtctacagactgaagctccgctgaaacgtgctcttatcccgttcggtggtatcaaaatgatcgagggttcctgcaaagcgtacaaccgcgaactggacccgatgatcaaaaaaatcttcactgaataccgtaaaactcacaaccagggcgtgttcgacgtttacactccggacatcctgcgttgccgtaaatccggtgttctgaccggtctgccagatgcttatggccgtggccgtatcatcggtgactaccgtcgcgttgcgctgtacggtatcgactacctgatgaaagacaaatacgctcagttcacctctctgcaggctgatctggaaaacggcgtaaacctggaacagactatccgtctgcgcgaagaaatcgctgaacagcaccgcgctctgggtcagatgaaagaaatggctgcgaaatacggctacgacatctctggtccggctaccaacgctcaggaagctatccagtggacttacttcggctacctggctgctgttaagtctcagaacggtgctgcaatgtccttcggtcgtacctccaccttcctggatgtgtacatcgaacgtgacctgaaagctggcaagatcaccgaacaagaagcgcaggaaatggttgaccacctggtcatgaaactgcgtatggttcgcttcctgcgtactccggaatacgatgaactgttctctggcgacccgatctgggcaaccgaatctatcggtggtatgggcctcgacggtcgtaccctggttaccaaaaacagcttccgtttcctgaacaccctgtacaccatgggtccgtctccggaaccgaacatgaccattctgtggtctgaaaaactgccgctgaacttcaagaaattcgccgctaaagtgtccatcgacacctcttctctgcaatatgagaacgatgacctgatgcgtccggacttcaacaacgatgactacgctattgcttgctgcgtaagcccgatgatcgttggtaaacaaatgcagttcttcggtgcgcgtgcaaacctggcgaaaaccatgctgtacgcaatcaacggcggcgttgacgaaaaactgaaaatgcaggttggtccgaagtctgaaccgatcaaaggcgatgtcctgaactatgatgaagtgatggagcgcatggatcacttcatggactggctggctaaacagtacatcactgcactgaacatcatccactacatgcacgacaagtacagctacgaagcctctctgatggcgctgcacgaccgtgacgttatccgcaccatggcgtgtggtatcgctggtctgtccgttgctgctgactccctgtctgcaatcaaatatgcgaaagttaaaccgattcgtgacgaagacggtctggctatcgacttcgaaatcgaaggcgaatacccgcagtttggtaacaatgatccgcgtgtagatgacctggctgttgacctggtagaacgtttcatgaagaaaattcagaaactgcacacctaccgtgacgctatcccgactcagtctgttctgaccatcacttctaacgttgtgtatggtaagaaaactggtaacaccccagacggtcgtcgtgctggcgcgccgttcggaccgggtgctaacccgatgcacggtcgtgaccagaaaggtgctgtagcgtctctgacttccgttgctaaactaccgtttgcttacgctaaagatggtatctcctacaccttctctatcgttccgaacgcactgggtaaagacgacgaagttcgtaagaccaacctggctggtctgatggatggttacttccaccacgaagcatccatcgaaggtggtcagcacctgaacgttaacgtgatgaaccgtgaaatgctgctcgacgcgatggaaaacccggaaaaatatccgcagctgaccatccgtgtatctggctacgcagtacgtttcaactcgctgactaaagaacagcagcaggacgttattactcgtaccttcactcaatctatgtaa

SEQ ID NO: 19 - protein, 891 aa, Escherichia coli
MAVTNVAELNALVERVKKAQREYASFTQEQVDKIFRAAALAAADARIPLAKMAVAESGMGIVEDKVIKNHFASEYIYNAYKDEKTCGVLSEDDTFGTITIAEPIGIICGIVPTTNPTSTAIFKSLISLKTRNAIIFSPHPRAKDATNKAADIVLQAAIAAGAPKDLIGWIDQPSVELSNALMHHPDINLILATGGPGMVKAAYSSGKPAIGVGAGNTPVVIDETADIKRAVASVLMSKTFDNGVICASEQSVVVVDSVYDAVRERFATHGGYLLQGKELKAVQDVILKNGALNAAIVGQPAYKIAELAGFSVPENTKILIGEVTVVDESEPFAHEKLSPTLAMYRAKDFEDAVEKAEKLVAMGGIGHTSCLYTDQDNQPARVSYFGQKMKTARILINTPASQGGIGDLYNFKLAPSLTLGCGSWGGNSISENVGPKHLINKKTVAKRAENMLWHKLPKSIYFRRGSLPIALDEVITDGHKRALIVTDRFLFNNGYADQITSVLKAAGVETEVFFEVEADPTLSIVRKGAELANSFKPDVIIALGGGSPMDAAKIMWVMYEHPETHFEELALRFMDIRKRIYKFPKMGVKAKMIAVTTTSGTGSEVTPFAVVTDDATGQKYPLADYALTPDMAIVDANLVMDMPKSLCAFGGLDAVTHAMEAYVSVLASEFSDGQALQALKLLKEYLPASYHEGSKNPVARERVHSAATIAGIAFANAFLGVCHSMAHKLGSQFHIPHGLANALLICNVIRYNANDNPTKQTAFSQYDRPQARRRYAEIADHLGLSAPGDRTAAKIEKLLAWLETLKAELGIPKSIREAGVQEADFLANVDKLSEDAFDDQCTGANPRYPLISELKQILLDTYYGRDYVEGETAAKKEAAPAKAEKKAKKSA

SEQ ID NO: 20 - DNA, 2676 bp, Escherichia coli
atggctgttactaatgtcgctgaacttaacgcactcgtagagcgtgtaaaaaaagcccagcgtgaatatgccagtttcactcaagagcaagtagacaaaatcttccgcgccgccgctctggctgctgcagatgctcgaatcccactcgcgaaaatggccgttgccgaatccggcatgggtatcgtcgaagataaagtgatcaaaaaccactttgcttctgaatatatctacaacgcctataaagatgaaaaaacctgtggtgttctgtctgaagacgacacttttggtaccatcactatcgctgaaccaatcggtattatttgcggtatcgttccgaccactaacccgacttcaactgctatcttcaaatcgctgatcagtctgaagacccgtaacgccattatcttctccccgcacccgcgtgcaaaagatgccaccaacaaagcggctgatatcgttctgcaggctgctatcgctgccggtgctccgaaagatctgatcggctggatcgatcaaccttctgttgaactgtctaacgcactgatgcaccacccagacatcaacctgatcctcgcgactggtggtccgggcatggttaaagccgcatacagctccggtaaaccagctatcggtgtaggcgcgggcaacactccagttgttatcgatgaaactgctgatatcaaacgtgcagttgcatctgtactgatgtccaaaaccttcgacaacggcgtaatctgtgcttctgaacagtctgttgttgttgttgactctgtttatgacgctgtacgtgaacgttttgcaacccacggcggctatctgttgcagggtaaagagctgaaagctgttcaggatgttatcctgaaaaacggtgcgctgaacgcggctatcgttggtcagccagcctataaaattgctgaactggcaggcttctctgtaccagaaaacaccaagattctgatcggtgaagtgaccgttgttgatgaaagcgaaccgttcgcacatgaaaaactgtccccgactctggcaatgtaccgcgctaaagatttcgaagacgcggtagaaaaagcagagaaactggttgctatgggcggtatcggtcatacctcttgcctgtacactgaccaggataaccaaccggctcgcgtttcttacttcggtcagaaaatgaaaacggctcgtatcctgattaacaccccagcgtctcagggtggtatcggtgacctgtataacttcaaactcgcaccttccctgactctgggttgtggttcttggggtggtaactccatctctgaaaacgttggtccgaaacacctgatcaacaagaaaaccgttgctaagcgagctgaaaacatgttgtggcacaaacttccgaaatctatctacttccgccgtggctccctgccaatcgcgctggatgaagtgattactgatggccacaaacgtgcgctcatcgtgactgaccgcttcctgttcaacaatggttatgctgatcagatcacttccgtactgaaagcagcaggcgttgaaactgaagtcttcttcgaagtagaagcggacccgaccctgagcatcgttcgtaaaggtgcagaactggcaaactccttcaaaccagacgtgattatcgcgctgggtggtggttccccgatggacgccgcgaagatcatgtgggttatgtacgaacatccggaaactcacttcgaagagctggcgctgcgctttatggatatccgtaaacgtatctacaagttcccgaaaatgggcgtgaaagcgaaaatgatcgctgtcaccaccacttctggtacaggttctgaagtcactccgtttgcggttgtaactgacgacgctactggtcagaaatatccgctggcagactatgcgctgactccggatatggcgattgtcgacgccaacctggttatggacatgccgaagtccctgtgtgctttcggtggtctggacgcagtaactcacgccatggaagcttatgtttctgtactggcatctgagttctctgatggtcaggctctgcaggcactgaaactgctgaaagaatatctgccagcgtcctaccacgaagggtctaaaaatccggtagcgcgtgaacgtgttcacagtgcagcgactatcgcgggtatcgcgtttgcgaacgccttcctgggtgtatgtcactcaatggcgcacaaactgggttcccagttccatattccgcacggtctggcaaacgccctgctgatttgtaacgttattcgctacaatgcgaacgacaacccgaccaagcagactgcattcagccagtatgaccgtccgcaggctcgccgtcgttatgctgaaattgccgaccacttgggtctgagcgcaccgggcgaccgtactgctgctaagatcgagaaactgctggcatggctggaaacgctgaaagctgaactgggtattccgaaatctatccgtgaagctggcgttcaggaagcagacttcctggcgaacgtggataaactgtctgaagatgcgttcgatgaccagtgcaccggcgctaacccgcgttacccgctgatctccgagctgaaacagatcctgctggatacctactacggtcgtgattatgtagaaggtgaaactgcagcgaaaaaagaagccgctccggctaaagctgagaaaaaagcgaaaaaatccgcttaa

SEQ ID NO: 21 - protein, 312 aa, Escherichia coli
MKVAVLGAAGGIGQALALLLKTQLPSGSELSLYDIAPVTPGVAVDLSHIPTAVKIKGFSGEDATPALEGADVVLISAGVARKPGMDRSDLFNVNAGIVKNLVQQVSKTCPKACIGIITNPVNTTVAIAAEVLKKAGVYDKNKLFGVTTLDIIRSNTFVAELKGKQPGEVEVPVIGGHSGVTILPLLSQVPGVSFTEQEVADLTKRIQNAGTEVVEAKAGGGSATLSMGQAAARFGLSLVRALQGEQGVVECAYVEGDGQYARFFSQPLLLGKNGVEERKSIGTLSAFEQSALEGMLDTLKKDIALGEEFVNK

SEQ ID NO: 22 - DNA, 939 bp, Escherichia coli
atgaaagtcgcagtcctcggcgctgctggcggtattggccaggcgcttgcactactgttaaaaacccaactgccttcaggttcagaactctctctgtatgatatcgctccagtgactcccggtgtggctgtcgatctgagccatatccctactgctgtgaaaatcaaaggtttttctggtgaagatgcgactccggcgctggaaggcgcagatgtcgttcttatctctgcaggtgtagcgcgtaaaccgggtatggatcgttccgacctgtttaacgttaacgccggcatcgtgaaaaacctggtacagcaagtttcgaaaacctgcccgaaagcgtgcattggtattatcactaacccggttaacaccacagttgcgattgctgctgaagtgctgaaaaaagccggtgtttatgacaaaaacaaactgttcggcgttaccacgctggatatcattcgttccaacacctttgttgcggaactgaaaggcaaacagccaggcgaagttgaagtgccggttattggcggtcactctggtgttaccattctgccgctgctgtcacaggttcctggcgttagttttaccgagcaggaagtggctgatctgaccaaacgtatccagaacgcaggtactgaagtggttgaagcgaaagccggtggcgggtctgcaaccctgtctatgggccaggcagctgcacgttttggtctgtctctggtacgcgcactgcagggcgaacaaggcgttgtcgaatgtgcctatgttgaaggcgacggtcagtacgcacgtttcttctctcaaccgctgctgctgggtaaaaacggcgtggaagagcgtaaatctatcggtaccctgagcgcatttgaacagagcgcactggaaggtatgctggatacgctgaagaaagatatcgccctgggcgaagagttcgttaataagtaa

SEQ ID NO: 23 - protein, 238 aa, Escherichia coli
MQTPHILIVEDELVTRNTLKSIFEAEGYDVFEATDGAEMHQILSEYDINLVIMDINLPGKNGLLLARELREQANVALMFLTGRDNEVDKILGLEIGADDYITKPFNPRELTIRARNLLSRTMNLGTVSEERRSVESYKFNGWELDINSRSLIGPDGEQYKLPRSEFRAMLHFCENPGKIQSRAELLKKMTGRELKPHDRTVDVTIRRIRKHFESTPDTPEIIATIHGEGYRFCGDLED

SEQ ID NO: 24 - DNA, 717 bp, Escherichia coli
atgcagaccccgcacattcttatcgttgaagacgagttggtaacacgcaacacgttgaaaagtattttcgaagcggaaggctatgatgttttcgaagcgacagatggcgcggaaatgcatcagatcctctctgaatatgacatcaacctggtgatcatggatatcaatctgccgggtaagaacggtcttctgttagcgcgtgaactgcgcgagcaggcgaatgttgcgttgatgttcctgactggccgtgacaacgaagtcgataaaattctcggcctcgaaatcggtgcagatgactacatcaccaaaccgttcaacccgcgtgaactgacgattcgtgcacgcaacctgctgtcccgtaccatgaatctgggtactgtcagcgaagaacgtcgtagcgttgaaagctacaagttcaatggttgggaactggatatcaacagccgttcgttgatcggccctgatggcgagcagtacaagctgccgcgcagcgagttccgcgccatgcttcacttctgtgaaaacccaggcaaaattcagtctcgtgctgaactgctgaagaaaatgaccggccgtgagctgaaaccacacgaccgtactgtagacgtgacgatccgccgtattcgtaaacatttcgaatctacgccggatacgccggaaatcatcgccaccatccacggtgaaggttatcgcttctgtggtgatctggaagattaa

SEQ ID NO: 25 - protein, 462 aa, Escherichia coli
MTITPATHAISINPATGEQLSVLPWAGANDIENALQLAAAGFRDWRETNIDYRAEKLRGIGKALRARSEEMAQMITREMGKPINQARAEVAKSANLCDWYAEHGPAMLKAEPTLVENQQAVIEYRPLGTILAIMPWNFPLWQVMRGAVPIILAGNGYLLKHAPNVMGCAQLIAQVFKDAGIPQGVYGWLNADNDGVSQMIKDSRIAAVTVTGSVRAGAAIGAQAGAALKKCVLELGGSDPFIVLNDADLELAVKAAVAGRYQNTGQVCAAAKRFIIEEGIASAFTERFVAAAAALKMGDPRDEENALGPMARFDLRDELHHQVEKTLAQGARLLLGGEKMAGAGNYYPPTVLANVTPEMTAFREEMFGPVAAITVAKDAEHALELANDSEFGLSATIFTTDETQARQMAARLECGGVFINGYCASDARVAFGGVKKSGFGRELSHFGLHEFCNIQTVWKDRI

SEQ ID NO: 26 - DNA, 1389 bp, Escherichia coli
atgaccattactccggcaactcatgcaatttcgataaatcctgccacgggtgaacaactttctgtgctgccgtgggctggcgctaacgatatcgaaaacgcacttcagctggcggcagcaggctttcgcgactggcgcgagacaaatatagattatcgtgctgaaaaactgcgtggtatcggtaaggctctgcgcgcccgtagcgaagaaatggcgcaaatgatcacccgtgaaatgggcaaaccaatcaatcaggcgcgcgctgaagtggcgaaatcggcgaatttgtgtgactggtatgcagaacatggtccggcaatgctgaaggcggaacctacgctggtggaaaatcagcaggcagttattgagtatcgaccgttggggacgattctggcgattatgccgtggaactttccgttatggcaggtgatgcgtggcgcggttcccatcattcttgcaggtaacggctacttacttaaacatgcgccgaatgtgatgggctgtgctcagctcattgcccaggtgtttaaagatgcgggaatcccgcaaggcgtatatggctggctgaatgccgacaacgacggtgtcagtcaaatgattaaagattcgcgcattgctgctgtcacggtgaccggaagtgttcgtgcgggagcggctattggcgcacaggctggagcggcactgaaaaaatgcgtactggaactgggcggttcggatccatttattgtgcttaacgatgccgatctggaactggcggttaaagcggcggtagccggacgttatcagaataccggacaggtttgtgcagcggcaaaacgctttattatcgaagagggaattgcttctgcatttaccgaacgttttgtggcagctgcggcagccttgaaaatgggcgatccccgtgatgaagagaacgctctcggaccaatggctcgttttgatttacgtgatgagctgcatcatcaggtggagaaaaccctggcgcagggtgcgcgtttgttactgggcggggaaaagatggctggggcaggtaattactatccgccaacggttctggcgaatgttaccccagaaatgaccgcgtttcgggaagaaatgtttggccctgttgcggcaatcaccgttgcgaaagatgcagaacatgcgctggaactggctaatgatagtgagttcggcctttcagcgaccatttttaccaccgacgaaacacaggccagacagatggcggcacgtctggaatgcggtggggtgtttatcaatggttattgtgccagcgacgcgcgagtggcctttggtggcgtgaaaaagagtggctttggtcgtgagctttcccatttcggcttacacgaattctgtaatatccagacggtgtggaaagaccggatctga

SEQ ID NO: 27 - protein, 482 aa, Escherichia coli
MKLNDSNLFRQQALINGEWLDANNGEVIDVTNPANGDKLGSVPKMGADETRAAIDAANRALPAWRALTAKERANILRNWFNLMMEHQDDLARLMTLEQGKPLAEAKGEISYAASFIEWFAEEGKRIYGDTIPGHQADKRLIVIKQPIGVTAAITPWNFPAAMITRKAGPALAAGCTMVLKPASQTPFSALALAELAIRAGIPAGVFNVVTGSAGAVGNELTSNPLVRKLSFTGSTEIGRQLMEQCAKDIKKVSLELGGNAPFIVFDDADLDKAVEGALASKFRNAGQTCVCANRLYVQDGVYDRFAEKLQQAVSKLHIGDGLDKGVTIGPLIDEKAVAKVEEHIADALEKGARVVCGGKAHERGGNFFQPTILVDVPANAKVSKEETFGPLAPLFRFKDEADVIAQANDTEFGLAAYFYARDLSRVFRVGEALEYGIVGINTGIISNEVAPFGGIKASGLGREGSKYGIEDYLEIKYMCIGL

SEQ ID NO: 28 - DNA, 1449 bp, Escherichia coli
atgaaacttaacgacagtaacttattccgccagcaggcgttgattaacggggaatggctggacgccaacaatggcgaggtcatcgacgtcaccaatccggcgaacggcgacaagctgggtagcgtacccaaaatgggcgctgatgaaacccgcgccgctatcgacgccgccaaccgcgctctgcccgcctggcgtgcgctcaccgccaaagaacgcgccaacattctgcgcaactggttcaatttgatgatggagcatcaggacgatttagcgcgtctgatgaccctcgaacagggtaaaccgctggctgaagcgaaaggtgaaatcagctacgccgcctcctttattgagtggtttgctgaagaaggcaaacgcatttatggcgacaccattcccggtcatcaggccgataaacgcctgattgttatcaagcagccgattggcgttaccgccgccatcacgccgtggaacttcccggcggcgatgattacccgtaaagccggtccggcgctggcggcaggctgcacgatggtgctgaaacccgccagtcagacgccgttctctgcgctggcgctggcggagctggcgattcgcgcgggcattccggctggggtatttaacgtggtcaccggttcggcgggcgcagtcggtaacgaactgaccagcaacccgctggtgcgcaaactgtcgtttaccggttcgaccgaaattggccgccagttaatggaacaatgcgcgaaagacatcaaaaaagtgtcgctggagctcggcggcaacgcgccgtttatcgtctttgacgatgccgacctcgacaaagccgtggaaggcgcgctggcctcgaaattccgcaacgccgggcaaacctgcgtctgcgccaaccgtttatacgtgcaggacggcgtgtatgaccgctttgccgaaaaattgcagcaggcggtgagcaaactgcacatcggcgacgggctggataaaggcgtcaccatcgggccgctgatcgatgaaaaagcagtagcaaaagtggaagagcatattgccgatgcgctggagaaaggcgcgcgcgtggtttgcggcggtaaagcacacgaacgtggcggcaacttcttccagccgaccattctggtggacgttccggccaacgctaaagtgtcgaaagaagagacgttcggccccctcgccccgctgttccgttttaaagatgaagccgatgtgatcgcgcaagccaatgacaccgaatttggtcttgccgcctatttctacgcccgtgatttaagccgcgtcttccgcgtgggcgaagcgctggagtacggcatcgtcggcatcaataccgggattatttccaatgaagtggccccgttcggcggcatcaaagcctcgggtctgggtcgtgaaggttcgaagtatggcatcgaagattacttagaaatcaaatatatgtgcatcggtctttaa

SEQ ID NO: 29 - protein, 474 aa, Klebsiella pneumoniae
MSTEIKTQVVVLGAGPAGYSAAFRCADLGLETVIVERYSTLGGVCLNVGCIPSKALLHVAKVIEEAKALAEHGIVFGEPKTDIDKIRTWKEKVITQLTGGLAGMAKGRKVKVVNGLGKFTGANTLEVEGENGKTVINFDNAIIAAGSRPIQLPFIPHEDPRVWDSTDALELKSVPKRMLVMGGGIIGLEMGTVYHALGSEIDVVEMFDQVIPAADKDVVKVFTKRISKKFNLMLETKVTAVEAKEDGIYVSMEGKKAPAEAQRYDAVLVAIGRVPNGKNLDAGKAGVEVDDRGFIRVDKQMRTNVPHIFAIGDIVGQPMLAHKGVHEGHVAAEVISGLKHYFDPKVIPSIAYTEPEVAWVGLTEKEAKEKGISYETATFPWAASGRAIASDCADGMTKLIFDKETHRVIGGAIVGTNGGELLGEIGLAIEMGCDAEDIALTIHAHPTLHESVGLAAEVFEGSITDLPNAKAKKK

SEQ ID NO: 30 - DNA, 1425 bp, Klebsiella pneumoniae
atgagtactgaaatcaaaactcaggtcgtggtacttggggcaggccccgcaggttactctgcagccttccgttgcgctgatttaggtctggaaaccgtcatcgtagaacgttacagcaccctcggtggtgtttgtctgaacgtgggttgtatcccttctaaagcgctgctgcacgtggcaaaagttatcgaagaagcgaaagcgctggccgaacacggcatcgttttcggcgaaccgaaaactgacattgacaagatccgcacctggaaagaaaaagtcatcactcagctgaccggtggtctggctggcatggccaaaggtcgtaaagtgaaggtggttaacggtctgggtaaatttaccggcgctaacaccctggaagtggaaggcgaaaacggcaaaaccgtgatcaacttcgacaacgccatcatcgcggcgggttcccgtccgattcagctgccgtttatcccgcatgaagatccgcgcgtatgggactccaccgacgcgctggaactgaaatctgtaccgaaacgcatgctggtgatgggcggcggtatcatcggtctggaaatgggtaccgtataccatgcgctgggttcagagattgacgtggtggaaatgttcgaccaggttatcccggctgccgacaaagacgtggtgaaagtcttcaccaaacgcatcagcaagaaatttaacctgatgctggaaaccaaagtgactgccgttgaagcgaaagaagacggtatttacgtttccatggaaggtaaaaaagcaccggcggaagcgcagcgttacgacgcagtgctggtcgctatcggccgcgtaccgaatggtaaaaacctcgatgcaggtaaagctggcgtggaagttgacgatcgcggcttcatccgcgttgacaaacaaatgcgcaccaacgtgccgcacatctttgctatcggcgatatcgtcggtcagccgatgctggcgcacaaaggtgtccatgaaggccacgttgccgcagaagttatctccggtctgaaacactacttcgatccgaaagtgatcccatccatcgcctacactgaaccagaagtggcatgggtcggtctgaccgagaaagaagcgaaagagaaaggcatcagctacgaaaccgccaccttcccgtgggctgcttccggccgtgctatcgcttctgactgcgcagatggtatgaccaaactgatcttcgacaaagagacccaccgtgttatcggcggcgcgattgtcggcaccaacggcggcgagctgctgggtgagatcggcctggctatcgagatgggctgtgacgctgaagacatcgccctgaccatccacgctcacccgactctgcacgagtccgttggcctggcggcggaagtgttcgaaggcagcatcaccgacctgccaaacgccaaagcgaagaaaaagtaa

SEQ ID NO: 31 - protein, 474 aa, Artificial Sequence
  Synthetic (lpdA E354K aa)
MSTEIKTQVVVLGAGPAGYSAAFRCADLGLETVIVERYSTLGGVCLNVGCIPSKALLHVAKVIEEAKALAEHGIVFGEPKTDIDKIRTWKEKVITQLTGGLAGMAKGRKVKVVNGLGKFTGANTLEVEGENGKTVINFDNAIIAAGSRPIQLPFIPHEDPRVWDSTDALELKSVPKRMLVMGGGIIGLEMGTVYHALGSEIDVVEMFDQVIPAADKDVVKVFTKRISKKFNLMLETKVTAVEAKEDGIYVSMEGKKAPAEAQRYDAVLVAIGRVPNGKNLDAGKAGVEVDDRGFIRVDKQMRTNVPHIFAIGDIVGQPMLAHKGVHEGHVAAEVISGLKHYFDPKVIPSIAYTKPEVAWVGLTEKEAKEKGISYETATFPWAASGRAIASDCADGMTKLIFDKETHRVIGGAIVGTNGGELLGEIGLAIEMGCDAEDIALTIHAHPTLHESVGLAAEVFEGSITDLPNAKAKKK

SEQ ID NO: 32 - DNA, 1425 bp, Artificial Sequence
  Synthetic (lpdA E354K nt)
atgagtactgaaatcaaaactcaggtcgtggtacttggggcaggccccgcaggttactctgcagccttccgttgcgctgatttaggtctggaaaccgtcatcgtagaacgttacagcaccctcggtggtgtttgtctgaacgtgggttgtatcccttctaaagcgctgctgcacgtggcaaaagttatcgaagaagcgaaagcgctggccgaacacggcatcgttttcggcgaaccgaaaactgacattgacaagatccgcacctggaaagaaaaagtcatcactcagctgaccggtggtctggctggcatggccaaaggtcgtaaagtgaaggtggttaacggtctgggtaaatttaccggcgctaacaccctggaagtggaaggcgaaaacggcaaaaccgtgatcaacttcgacaacgccatcatcgcggcgggttcccgtccgattcagctgccgtttatcccgcatgaagatccgcgcgtatgggactccaccgacgcgctggaactgaaatctgtaccgaaacgcatgctggtgatgggcggcggtatcatcggtctggaaatgggtaccgtataccatgcgctgggttcagagattgacgtggtggaaatgttcgaccaggttatcccggctgccgacaaagacgtggtgaaagtcttcaccaaacgcatcagcaagaaatttaacctgatgctggaaaccaaagtgactgccgttgaagcgaaagaagacggtatttacgtttccatggaaggtaaaaaagcaccggcggaagcgcagcgttacgacgcagtgctggtcgctatcggccgcgtaccgaatggtaaaaacctcgatgcaggtaaagctggcgtggaagttgacgatcgcggcttcatccgcgttgacaaacaaatgcgcaccaacgtgccgcacatctttgctatcggcgatatcgtcggtcagccgatgctggcgcacaaaggtgtccatgaaggccacgttgccgcagaagttatctccggtctgaaacactacttcgatccgaaagtgatcccatccatcgcctacactaagccagaagtggcatgggtcggtctgaccgagaaagaagcgaaagagaaaggcatcagctacgaaaccgccaccttcccgtgggctgcttccggccgtgctatcgcttctgactgcgcagatggtatgaccaaactgatcttcgacaaagagacccaccgtgttatcggcggcgcgattgtcggcaccaacggcggcgagctgctgggtgagatcggcctggctatcgagatgggctgtgacgctgaagacatcgccctgaccatccacgctcacccgactctgcacgagtccgttggcctggcggcggaagtgttcgaaggcagcatcaccgacctgccaaacgccaaagcgaagaaaaagtaa

SEQ ID NO: 33 - protein, 427 aa, Escherichia coli
MADTKAKLTLNGDTAVELDVLKGTLGQDVIDIRTLGSKGVFTFDPGFTSTASCESKITFIDGDEGILLHRGFPIDQLATDSNYLEVCYILLNGEKPTQEQYDEFKTTVTRHTMIHEQITRLFHAFRRDSHPMAVMCGITGALAAFYHDSLDVNNPRHREIAAFRLLSKMPTMAAMCYKYSIGQPFVYPRNDLSYAGNFLNMMFSTPCEPYEVNPILERAMDRILILHADHEQNASTSTVRTAGSSGANPFACIAAGIASLWGPAHGGANEAALKMLEEISSVKHIPEFVRRAKDKNDSFRLMGFGHRVYKNYDPRATVMRETCHEVLKELGTKDDLLEVAMELENIALNDPYFIEKKLYPNVDFYSGIILKAMGIPSSMFTVIFAMARTVGWIAHWSEMHSDGMKIARPRQLYTGYEKRDFKSDIKR

SEQ ID NO: 34 - DNA, 1284 bp, Escherichia coli
atggctgatacaaaagcaaaactcaccctcaacggggacacagctgttgaactggatgtgctgaaaggcacgctgggtcaagatgttattgatatccgtactctcggttcaaaaggtgtgttcacctttgacccaggcttcacttcaaccgcatcctgcgaatctaaaattacttttattgatggtgatgaaggtattttgctgcaccgcggtttcccgatcgatcagctggcgaccgattctaactacctggaagtttgttacatcctgctgaatggtgaaaaaccgactcaggaacagtatgacgaatttaaaactacggtgacccgtcataccatgatccacgagcagattacccgtctgttccacgctttccgtcgcgactcacatccaatggcagtcatgtgtggtattaccggcgcgctggcggcgttctatcacgactcgctggatgttaacaatcctcgtcatcgtgaaattgccgcgttccgcctgctgtcgaaaatgccgaccatggccgcgatgtgttacaagtattccattggtcagccatttgtttatccgcgcaacgatctctcctatgccggtaacttcctgaatatgatgttctccacgccgtgcgaaccgtatgaagttaatccgattctggaacgtgctatggaccgtattctgatcctgcacgctgaccatgaacagaacgcctctacctccaccgtgcgtaccgctggctcttcgggtgcgaacccgtttgcctgtatcgcagcaggtattgcttcactgtggggacctgcgcacggtggtgctaacgaagcggcgctgaaaatgctggaagaaattagctccgttaaacacattccggaatttgttcgtcgtgcgaaagataaaaatgattctttccgcctgatgggcttcggtcaccgcgtgtacaaaaattacgacccgcgcgccaccgtaatgcgtgaaacctgccatgaagttctgaaagagctgggcaccaaagatgacctgctggaagtggctatggagctggaaaacatcgcgctgaacgacccgtactttatcgagaagaaactgtacccgaacgtcgatttctactctggtatcatcctgaaagcgatgggtattccgtcttccatgttcaccgtcattttcgcaatggcacgtaccgttggctggatcgcccactggagcgaaatgcacagtgacggtatgaagattgcccgtccgcgtcagctgtatacaggatatgaaaaacgcgactttaaaagcgatatcaagcgttaa

SEQ ID NO: 35 - protein, 427 aa, Artificial Sequence
  Synthetic (gltA R164L aa)
MADTKAKLTLNGDTAVELDVLKGTLGQDVIDIRTLGSKGVFTFDPGFTSTASCESKITFIDGDEGILLHRGFPIDQLATDSNYLEVCYILLNGEKPTQEQYDEFKTTVTRHTMIHEQITRLFHAFRRDSHPMAVMCGITGALAAFYHDSLDVNNPRHREIAAFLLLSKMPTMAAMCYKYSIGQPFVYPRNDLSYAGNFLNMMFSTPCEPYEVNPILERAMDRILILHADHEQNASTSTVRTAGSSGANPFACIAAGIASLWGPAHGGANEAALKMLEEISSVKHIPEFVRRAKDKNDSFRLMGFGHRVYKNYDPRATVMRETCHEVLKELGTKDDLLEVAMELENIALNDPYFIEKKLYPNVDFYSGIILKAMGIPSSMFTVIFAMARTVGWIAHWSEMHSDGMKIARPRQLYTGYEKRDFKSDIKR

SEQ ID NO: 36 - DNA, 1284 bp, Artificial Sequence
  Synthetic (gltA R164L nt)
atggctgatacaaaagcaaaactcaccctcaacggggacacagctgttgaactggatgtgctgaaaggcacgctgggtcaagatgttattgatatccgtactctcggttcaaaaggtgtgttcacctttgacccaggcttcacttcaaccgcatcctgcgaatctaaaattacttttattgatggtgatgaaggtattttgctgcaccgcggtttcccgatcgatcagctggcgaccgattctaactacctggaagtttgttacatcctgctgaatggtgaaaaaccgactcaggaacagtatgacgaatttaaaactacggtgacccgtcataccatgatccacgagcagattacccgtctgttccacgctttccgtcgcgactcacatccaatggcagtcatgtgtggtattaccggcgcgctggcggcgttctatcacgactcgctggatgttaacaatcctcgtcatcgtgaaattgccgcgttcctcctgctgtcgaaaatgccgaccatggccgcgatgtgttacaagtattccattggtcagccatttgtttatccgcgcaacgatctctcctatgccggtaacttcctgaatatgatgttctccacgccgtgcgaaccgtatgaagttaatccgattctggaacgtgctatggaccgtattctgatcctgcacgctgaccatgaacagaacgcctctacctccaccgtgcgtaccgctggctcttcgggtgcgaacccgtttgcctgtatcgcagcaggtattgcttcactgtggggacctgcgcacggtggtgctaacgaagcggcgctgaaaatgctggaagaaattagctccgttaaacacattccggaatttgttcgtcgtgcgaaagataaaaatgattctttccgcctgatgggcttcggtcaccgcgtgtacaaaaattacgacccgcgcgccaccgtaatgcgtgaaacctgccatgaagttctgaaagagctgggcaccaaagatgacctgctggaagtggctatggagctggaaaacatcgcgctgaacgacccgtactttatcgagaagaaactgtacccgaacgtcgatttctactctggtatcatcctgaaagcgatgggtattccgtcttccatgttcaccgtcattttcgcaatggcacgtaccgttggctggatcgcccactggagcgaaatgcacagtgacggtatgaagattgcccgtccgcgtcagctgtatacaggatatgaaaaacgcgactttaaaagcgatatcaagcgttaa

SEQ ID NO: 37 - DNA, 71 bp, Artificial Sequence
  Synthetic (ldhA KO f)
atgaaactcgccgtttatagcacaaaacagtacgacaagaagtacctgcataggtgacactatagaacgcg

SEQ ID NO: 38 - DNA, 70 bp, Artificial Sequence
  Synthetic (ldhA KO r)
ttaaaccagttcgttcgggcaggtttcgcctttttccagattgcttaagttagtggatctgatgggtacc

SEQ ID NO: 39 - DNA, 20 bp, Artificial Sequence
  Synthetic (ldhA KO CUP)
tacactaagcatagttgttg

SEQ ID NO: 40 - DNA, 20 bp, Artificial Sequence
  Synthetic (ldhA KO CDO)
ctttcttcattgtggttctc

SEQ ID NO: 41 - DNA, 71 bp, Artificial Sequence
  Synthetic (pflB KO f)
atgtccgagcttaatgaaaagttagccacagcctgggaaggttttaccaataggtgacactatagaacgcg

SEQ ID NO: 42 - DNA, 70 bp, Artificial Sequence
  Synthetic (pflB KO r)
ttacatagattgagtgaaggtacgagtaataacgtcctgctgctgttctttagtggatctgatgggtacc

SEQ ID NO: 43 - DNA, 20 bp, Artificial Sequence
  Synthetic (pflB KO CUP)
gggtcatttacctgcgtgaa

SEQ ID NO: 44 - DNA, 20 bp, Artificial Sequence
  Synthetic (pflB KO CDO)
agtctgttttggcagtcacc

SEQ ID NO: 45 - DNA, 71 bp, Artificial Sequence
  Synthetic (adhE KO f)
atggctgttactaatgtcgctgaacttaacgcactcgtagagcgtgtaaataggtgacactatagaacgcg

SEQ ID NO: 46 - DNA, 70 bp, Artificial Sequence
  Synthetic (adhE KO r)
ttaagcggattttttcgcttttttctcagctttagccggagcggcttctttagtggatctgatgggtacc

SEQ ID NO: 47 - DNA, 20 bp, Artificial Sequence
  Synthetic (adhE KO CUP)
caccgcactgactatactct

SEQ ID NO: 48 - DNA, 20 bp, Artificial Sequence
  Synthetic (adhE KO CDO)
gatgaaggctaatgctgtcg

SEQ ID NO: 49 - DNA, 71 bp, Artificial Sequence
  Synthetic (mdh KO f)
atgaaagtcgcagtcctcggcgctgctggcggtattggccaggcgcttgctaggtgacactatagaacgcg

SEQ ID NO: 50 - DNA, 70 bp, Artificial Sequence
  Synthetic (mdh KO r)
ttacttattaacgaactcttcgcccagggcgatatctttcttcagcgtattagtggatctgatgggtacc

SEQ ID NO: 51 - DNA, 20 bp, Artificial Sequence
  Synthetic (mdh KO CUP)
ggttcctgattacggcaatt

SEQ ID NO: 52 - DNA, 20 bp, Artificial Sequence
  Synthetic (mdh KO CDO)
attcaggaatatccggcaac

SEQ ID NO: 53 - DNA, 71 bp, Artificial Sequence
  Synthetic (arcA KO f)
atgcagaccccgcacattcttatcgttgaagacgagttggtaacacgcaataggtgacactatagaacgcg

SEQ ID NO: 54 - DNA, 70 bp, Artificial Sequence
  Synthetic (arcA KO r)
ttaatcttccagatcaccacagaagcgataaccttcaccgtggatggtggtagtggatctgatgggtacc

SEQ ID NO: 55 - DNA, 20 bp, Artificial Sequence
  Synthetic (arcA KO CUP)
ttgacgttgatggaaagtgc

SEQ ID NO: 56 - DNA, 20 bp, Artificial Sequence
  Synthetic (arcA KO CDO)
ccgaaaatgaaagccagtaa

SEQ ID NO: 57 - DNA, 71 bp, Artificial Sequence
  Synthetic (sad KO f)
atgaccattactccggcaactcatgcaatttcgataaatcctgccacgggtaggtgacactatagaacgcg

SEQ ID NO: 58 - DNA, 70 bp, Artificial Sequence
  Synthetic (sad KO r)
tcagatccggtctttccacaccgtctggatattacagaattcgtgtaagctagtggatctgatgggtacc

SEQ ID NO: 59 - DNA, 20 bp, Artificial Sequence
  Synthetic (sad KO CUP)
tcgattcgtgaataagtggc

SEQ ID NO: 60 - DNA, 20 bp, Artificial Sequence
  Synthetic (sad KO CDO)
ccactttctactcctggacc

SEQ ID NO: 61 - DNA, 71 bp, Artificial Sequence
  Synthetic (gabD KO f)
atgaaacttaacgacagtaacttattccgccagcaggcgttgattaacggtaggtgacactatagaacgcg

SEQ ID NO: 62 - DNA, 70 bp, Artificial Sequence
  Synthetic (gabD KO r)
ttaaagaccgatgcacatatatttgatttctaagtaatcttcgatgccattagtggatctgatgggtacc

SEQ ID NO: 63 - DNA, 20 bp, Artificial Sequence
  Synthetic (gabD KO CUP)
cacgccgcatttaatcaata

SEQ ID NO: 64 - DNA, 20 bp, Artificial Sequence
  Synthetic (gabD KO CDO)
ctctttattgctgctcattc

SEQ ID NO: 65 - DNA, 28 bp, Artificial Sequence
  Synthetic (K lpd F)
ccatcgcctacactaagccagaagtggc

SEQ ID NO: 66 - DNA, 28 bp, Artificial Sequence
  Synthetic (K lpd R)
gccacttctggcttagtgtaggcgatgg

SEQ ID NO: 67 - DNA, 82 bp, Artificial Sequence
  Synthetic (K lpd 3)
gccgctgcggcctgaaagacgacgggtatgaccgccggagataaatatatagaggtcatgaactgtctgcttacataaacag

SEQ ID NO: 68 - DNA, 77 bp, Artificial Sequence
  Synthetic (K lpd 4)
taaaaaaagcggcgtggttagccgcttttttaattgccggatgttccggcaaacgaacaattggtcggtcatttcgc

SEQ ID NO: 69 - DNA, 90 bp, Artificial Sequence
  Synthetic (K lpd 5)
ccggatccgccgctgcggcctgaaagacgacgggtatgaccgccggagataaatatatagaggtcatgatgagtactgaaatcaaaactc

SEQ ID NO: 70 - DNA, 89 bp, Artificial Sequence
  Synthetic (K lpd 6)
gggtcgactaaaaaaagcggcgtggttagccgcttttttaattgccggatgttccggcaaacgaacaattactttttcttcgctttggc

SEQ ID NO: 71 - DNA, 19 bp, Artificial Sequence
  Synthetic (K lpd 7)
catcattaacaacacgctg

SEQ ID NO: 72 - DNA, 19 bp, Artificial Sequence
  Synthetic (K lpd 8)
cgacagtaaccatactgtc

SEQ ID NO: 73 - DNA, 17 bp, Artificial Sequence
  Synthetic (gltA F)
tcgacagcaggaggaac

SEQ ID NO: 74 - DNA, 17 bp, Artificial Sequence
  Synthetic (gltA R)
tcgacagcaggaggaac

SEQ ID NO: 75 - DNA, 80 bp, Artificial Sequence
  Synthetic (gltA 3)
gtgcgaaggcaaatttaagttccggcagtcttacgtaataaggcgctaaggagaccttaactgtctgcttacataaacag

SEQ ID NO: 76 - DNA, 78 bp, Artificial Sequence
  Synthetic (gltA 4)
ataaaaattaacccgccatttgaacggcgggttaaaatatttacaacttagcaatcaaccattggtcggtcatttcgc

SEQ ID NO: 77 - DNA, 67 bp, Artificial Sequence
  Synthetic (gltA 5)
gtgcgaaggcaaatttaagttccggcagtcttacgtaataaggcgctaaggagaccttaaatggctg

SEQ ID NO: 78 - DNA, 78 bp, Artificial Sequence
  Synthetic (gltA 6)
ataaaaattaacccgccatttgaacggcgggttaaaatatttacaacttagcaatcaaccattaacgcttgatatcgc

SEQ ID NO: 79 - DNA, 21 bp, Artificial Sequence
  Synthetic (gltA 7)
ggacagttattagtggtagac

SEQ ID NO: 80 - DNA, 22 bp, Artificial Sequence
  Synthetic (gltA 8)
gatgtatttcacacggtgcttc

SEQ ID NO: 81 - DNA, 55 bp, Artificial Sequence
  Synthetic (L273I primer f)
gaaggctgctcatttgataacaacatcccgtgcattgctgagaaagaagttttcg

SEQ ID NO: 82 - DNA, 55 bp, Artificial Sequence
  Synthetic (L273I primer r)
cgaaaacttctttctcagcaatgcacgggatgttgttatcaaatgagcagccttc

SEQ ID NO: 83 - DNA, 55 bp, Artificial Sequence
  Synthetic (L273S primer f)
gaaggctgctcatttgataacaactccccgtgcattgctgagaaagaagttttcg

SEQ ID NO: 84 - DNA, 55 bp, Artificial Sequence
  Synthetic (L273S primer r)
cgaaaacttctttctcagcaatgcacggggagttgttatcaaatgagcagccttc